(19) United States
(12) Patent Application Publication
(10) Pub. No.: US 2021/0322022 A1
(43) Pub. Date: Oct. 21, 2021

(54) MEDICAL DEVICE AND METHOD OF INSTALLING MEDICAL DEVICE

(71) Applicant: OLYMPUS CORPORATION, Tokyo (JP)

(72) Inventors: Kensuke UESAKA, Tokyo (JP); Shinya ANSAI, Tokyo (JP); Ryu YORITA, Tokyo (JP); Naoki TAKIZAWA, Tokyo (JP)

(73) Assignee: OLYMPUS CORPORATION, Tokyo (JP)

(21) Appl. No.: 17/357,389

(22) Filed: Jun. 24, 2021

Related U.S. Application Data
(63) Continuation of application No. PCT/JP2018/048235, filed on Dec. 27, 2018.

(51) Int. Cl. A61B 17/128; A61B 17/122

(52) U.S. Cl. CPC A61B 17/128; A61B 17/122

(57) ABSTRACT

A medical device includes a clip having an arm member and a pressing tube configured to receive the arm member; an applicator having an operation wire configured to advance and retract to open and close the arm member and a sheath configured to accommodate the operation wire; and a link configured to engage the operation wire with the clip and transition between an engagement configuration in which the clip and the operation wire is engaged with each other and a release configuration in which the engagement of the clip and the operation wire is released.

This application is a continuation application of PCT International Application No. PCT/JP2018/048235, filed on Dec. 27, 2018. The content of the PCT International Applications is incorporated herein by reference.

### TECHNICAL FIELD

The present disclosure relates to a clip cartridge for accommodating a clip unit configured to treat a target tissue in a body, a medical device (more specifically, a ligation device used for ligating a tissue), and an attachment method for attaching the medical device.

### BACKGROUND ART

Conventionally, an endoscope treatment device introduced into the body of a patient through a channel of the endoscope for ligating the openings formed in the tissues and the blood vessels in the body is disclosed in Japanese Unexamined Patent Application, First Publication No. 2009-011852.

The endoscope treatment device disclosed in Japanese Unexamined Patent Application, First Publication No. 2009-011852 is configured by engaging a clip unit accommodated in a clip case and an operation wire inserted through an insertion portion of the endoscope with each other. According to the endoscope treatment device disclosed in Japanese Unexamined Patent Application, First Publication No. 2009-011852, the operation wire and the clip unit are engaged with each other by inserting a hook formed at the distal end side of the operation wire and in an arrowhead shape into a notch portion formed at the proximal end side of the clip unit. According to the endoscope treatment device disclosed in Japanese Unexamined Patent Application, First Publication No. 2009-011852, at the time when the operation wire and the clip unit are engaged with each other, the engagement portion of the hook and the connection member is positioned in the sheath of the insertion portion of the endoscope.

In the endoscope treatment device disclosed in Japanese Unexamined Patent Application, First Publication No. 2009-011852, a pair of arms of the clip unit are formed to be intersected with each other and part of the pair of arms are accommodated in the pressing tube. Accordingly, in the state in which the operation wire and the clip unit are engaged with each other, by the operation of retracting the operation wire to the proximal end side, the pair of arms are in contact with the pressing tube while the opening width between the pair of arms are increased once and then decreased to a degree suitable to tightly bind the target tissue.

According to the endoscope treatment device disclosed in Japanese Unexamined Patent Application, First Publication No. 2009-011852, the connection member disposed at the proximal end side of the clip unit has a small-diameter portion configured to be broken in a case in which a force exceeding the predetermined tensile strength is applied to the connection member. Accordingly, according to the endoscope treatment device disclosed in Japanese Unexamined Patent Application, First Publication No. 2009-011852, in the state in which the target tissue is grasped by the pair of arms of the clip unit, when the operator retracts the operation wire toward the proximal end side, the connection member is broken. In this manner, it is possible to indwell the clip unit grasping the target tissue in the body.

Generally, the medical device configured to perform treatment (ligation) with respect to the tissues inside the body is configured by connecting the treatment device (for example, the clip) inserted into the body and the applicator configured to adjust the position and the orientation of the treatment portion by the hand of the operator. When using the treatment device to perform treatment to the target tissues inside the body, for example, there is a case to further perform treatment to the new target tissues that are not discovered before the surgeon. At this time, for example, in the case of using the clip as the treatment device, it is necessary to exchange the plurality of clips so as to perform ligation to each of the plurality of target tissues. In other words, in the medical device for treating the target tissues inside the body, it is preferable to attach the new treatment device to the applicator, that is, it is preferable that the treatment device may be reloaded.

On the other hand, at the time of treating one target tissue inside the body, for example, in a case in which the tissue is unintentionally grasped by the clip used as the treatment device or in a case in which the state of grasping the target tissues is necessary to be adjusted, it is preferable to once release the grasping state of the tissues by the treatment device and then grasp the tissues again, that is, it is preferable to re-grasp the treatment device.

As disclosed above, in order to suitably perform treatment to the target tissues inside the body, it is preferable to achieve both goals of reloading the treatment portion and re-grasping the target tissues by the treatment device.

According to the endoscope treatment device disclosed in Japanese Unexamined Patent Application, First Publication No. 2009-011852, it is possible to cause the small-diameter portion in the connection member to be broken by retracting the operation wire toward the proximal end side in the state in which the target tissues are grasped by the clip unit so as to indwell the clip unit inside the body. Thereafter, by the operator pushing the operation wire toward the distal end side, it is possible to protrude the hook formed in the arrowhead shape and disposed at the distal end portion of the operation wire from the distal end opening of the sheath. Accordingly, it is possible to detach part of the connection member 3 engaged with the hook in the arrowhead shape and attach a different clip unit thereto, that is, it is possible to reload the clip.

According to the endoscope treatment device disclosed in Japanese Unexamined Patent Application, First Publication No. 2009-011852, at the time of performing the treatment to the target tissues inside the body, it is necessary to advance the clip unit to the distal end side by pushing the operation wire to the distal end side so as to adjust the opening width of the pair of arms of the clip. However, due to such movement, it is possible for the engagement portion of the hook and the connection member of the clip unit to move the position from which the engagement portion may protrude from the distal end opening of the sheath. Accordingly, it is possible that the engagement of the hook and the connection member is released. In other words, the endoscope treatment device disclosed in Japanese Unexamined Patent Application, First Publication No. 2009-011852 is configured without taking the configuration for re-grasping the target tissues by the treatment portion into consideration, and it is impossible to achieve the both goals of reloading the treatment portion and re-grasping the target tissues by the treatment device.

### SUMMARY

According to an aspect of the present disclosure, a medical device includes a clip unit having an arm member and a pressing tube into which the arm member is inserted; an operation wire configured to open and close the arm member by an advancement and retraction of the operation wire; a sheath configured to accommodate the operation wire; and a link mechanism configured to cause the operation wire and the clip unit to engage with each other, wherein the link mechanism has a convex portion configured to transition between a first configuration in which the convex portion has a first width in a predetermined direction around a longitudinal axis of the operation wire and a second configuration in which the convex portion has a second width larger than the first width in the predetermined direction; and an accommodation portion having an insertion port with an opening width larger than the first width and less than the second width in the predetermined direction around the longitudinal axis of the operation wire, wherein the convex portion is apart from an edge of the insertion port to form a space between the convex portion and the edge of the insertion port when the convex portion is inserted into the insertion port, and wherein the link mechanism is configured to transition to the second configuration so as to cause the convex portion and the accommodation portion to engage with each other when the convex portion passes through the insertion port in the first configuration.

According to another aspect of the present disclosure, an attachment method for attaching a clip unit in a medical device to an applicator, the clip unit having an arm member and a holding tube into which the arm member is inserted, and the arm member by moving back and forth. An applicator having an operation wire for opening and closing the operation wire and a sheath for accommodating the operation wire; and a release mode in which the operation wire and the clip unit are engaged, and the operation wire and the clip unit are disengaged. In a medical device including a link that transitions between the operation wire and the engagement form in which the clip unit is engaged, a mounting method for mounting the clip unit on the applicator, wherein the sheath is the pressing tube. And a step of maximally advancing the operating wire, the transition of the link from the released form to the engaged form inside the presser tube or the sheath.

### BRIEF DESCRIPTION OF DRAWINGS

FIG. 1 is a partial cross-sectional planar view schematically showing a configuration of a clip unit according to a first embodiment of the present disclosure.

FIG. 2 is a partial cross-sectional side view schematically showing the configuration of the clip unit according to the present embodiment.

FIG. 3A is a perspective view schematically showing a configuration of an intermediate portion of the clip unit according to the present embodiment.

FIG. 3B is a view showing the clip unit according to the present embodiment from the proximal end side.

FIG. 4A is a partial cross-sectional side view showing a configuration of an applicator according to the present embodiment.

FIG. 4B is a perspective view showing a distal end portion of an operation wire in the applicator according to the present embodiment.

FIG. 5 is a view showing an operation to attach the clip unit to the applicator according to the present embodiment.

FIG. 6 is a view showing the operation to attach the clip unit to the applicator according to the present embodiment.

FIG. 7 is a view showing the operation to attach the clip unit to the applicator according to the present embodiment.

FIG. 8A is a view showing the operation to attach the clip unit to the applicator according to the present embodiment.

FIG. 8B is a view showing the operation to attach the clip unit to the applicator according to the present embodiment.

FIG. 9 is a view showing an operation of treating the target tissues using the medical device according to the present embodiment.

FIG. 10 is a view showing an operation of treating the target tissues using the medical device according to the present embodiment.

FIG. 11A is a view showing an operation of treating the target tissues using the medical device according to the present embodiment.

FIG. 11B is a view showing an operation of treating the target tissues using the medical device according to the present embodiment.

FIG. 12A is a view showing an operation of treating the target tissues using the medical device according to the present embodiment.

FIG. 12B is a view showing an operation of treating the target tissues using the medical device according to the present embodiment.

FIG. 13 is a view showing an operation of treating the target tissues using the medical device according to the present embodiment.

FIG. 14A is a partial cross-sectional side view showing a configuration of the applicator according to a modification of the present embodiment.

FIG. 14B is a partial cross-sectional side view showing a configuration of the applicator according to a modification of the present embodiment.

FIG. 15 is a view showing an operation to attach the clip unit to the applicator according to the present modification.

FIG. 16 is a view showing an operation to attach the clip unit to the applicator according to the present modification.

FIG. 17 is a view showing an operation to attach the clip unit to the applicator according to the present modification.

FIG. 18A is a view showing an operation to attach the clip unit to the applicator according to a second embodiment of the present disclosure.

FIG. 18B is a view showing the operation to attach the clip unit to the applicator according to the present embodiment.

FIG. 18C is a view showing the operation to attach the clip unit to the applicator according to the present embodiment.

FIG. 18D is a view showing the operation to attach the clip unit to the applicator according to the present embodiment.

FIG. 19A is a view showing an operation to attach the clip unit to the applicator according to a third embodiment of the present disclosure.

FIG. 19B is a view showing the operation to attach the clip unit to the applicator according to the present embodiment.

FIG. 19C is a view showing the operation to attach the clip unit to the applicator according to a modification of the present embodiment.

FIG. 19D is a view showing the operation to attach the clip unit to the applicator according to the present modification.

FIG. 20A is a view showing the operation to attach the clip unit to the applicator according to the present embodiment.

FIG. 20B is a partial enlarged view showing a state in which the clip unit and the applicator shown in FIG. 20A are engaged with each other.

FIG. 20C is a view showing the operation to attach the clip unit to the applicator according to the present embodiment.

FIG. 20D is a partial enlarged view showing a state in which the clip unit and the applicator shown in FIG. 20C are engaged with each other.

FIG. 21 is a view showing the operation to attach the clip unit to the applicator according to the present embodiment.

FIG. 22 is a view showing an operation to perform treatment to the target tissues using the medical device according to the present embodiment.

FIG. 23 is a view showing the operation to perform treatment to the target tissues using the medical device according to the present embodiment.

FIG. 24 is a view showing the operation to perform treatment to the target tissues using the medical device a according to the present embodiment.

FIG. 25 is a view showing the operation to perform treatment to the target tissues using the medical device according to the present embodiment.

FIG. 26 is a view showing an operation to attach the clip unit to the applicator according to a fourth embodiment of the present disclosure.

FIG. 27 is a view showing the operation to attach the clip unit to the applicator according to the present embodiment.

FIG. 28 is a view showing the operation to attach the clip unit to the applicator according to the present embodiment.

FIG. 29 is a view showing an operation to attach the clip unit to the applicator according to a fifth embodiment of the present disclosure.

FIG. 30 is a view showing the operation to attach the clip unit to the applicator according to the present embodiment.

FIG. 31 is a view showing the operation to attach the clip unit to the applicator according to the present embodiment.

FIG. 32 is a view showing the operation to attach the clip unit to the applicator according to the present embodiment.

FIG. 33 is a view showing the operation to attach the clip unit to the applicator according to the present embodiment.

### DESCRIPTION OF EMBODIMENTS

### First embodiment

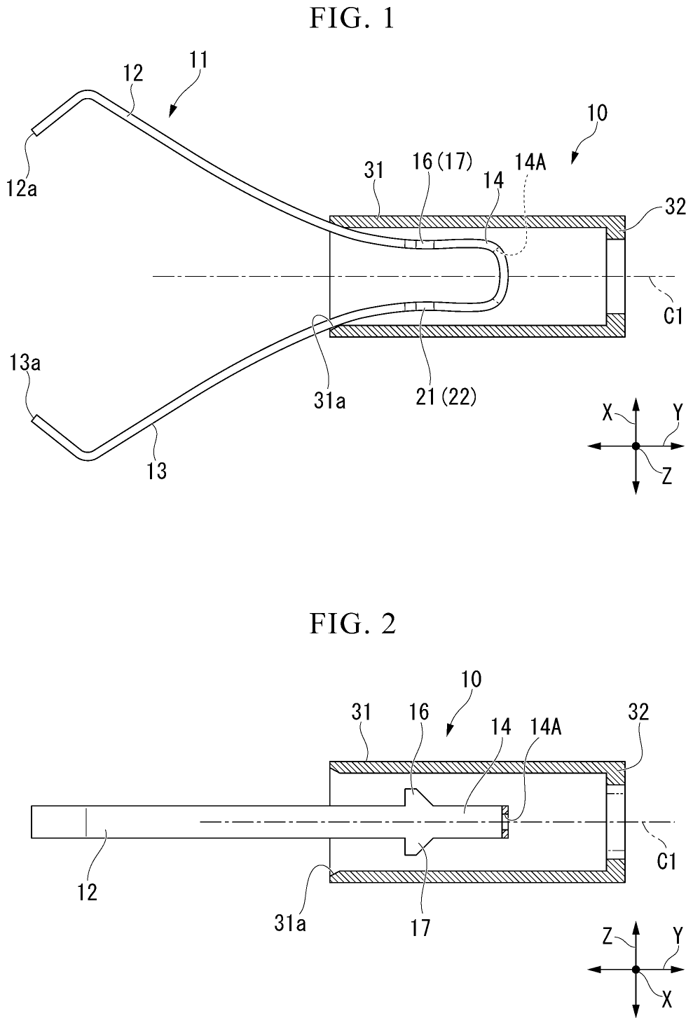

Hereinafter, a configuration of a medical device according to a first embodiment of the present disclosure will be described with reference to FIG. 1 to FIG. 4B.

The medical device 1 according to the present embodiment is used by being inserted into the body of a patient through a channel formed in an endoscope (not shown). More specifically, the medical device 1 according to the present embodiment is a ligation device for ligating a target tissue in the body.

In the present specification, the side on which the endoscope operation portion for the operator to operate the endoscope is located is defined as a proximal end side, and the side on which the distal end portion of the endoscope inserted into the body is located is defined as a distal end side. More specifically, the proximal end side of a medical device 1 according to the present embodiment is defined as the side on which the endoscope operation portion is located when the medical device 1 is inserted into a channel formed in the endoscope. The distal end side of the medical device 1 is defined as the side where the distal end portion of the endoscope is located when the medical device 1 is inserted into the channel formed in the endoscope.

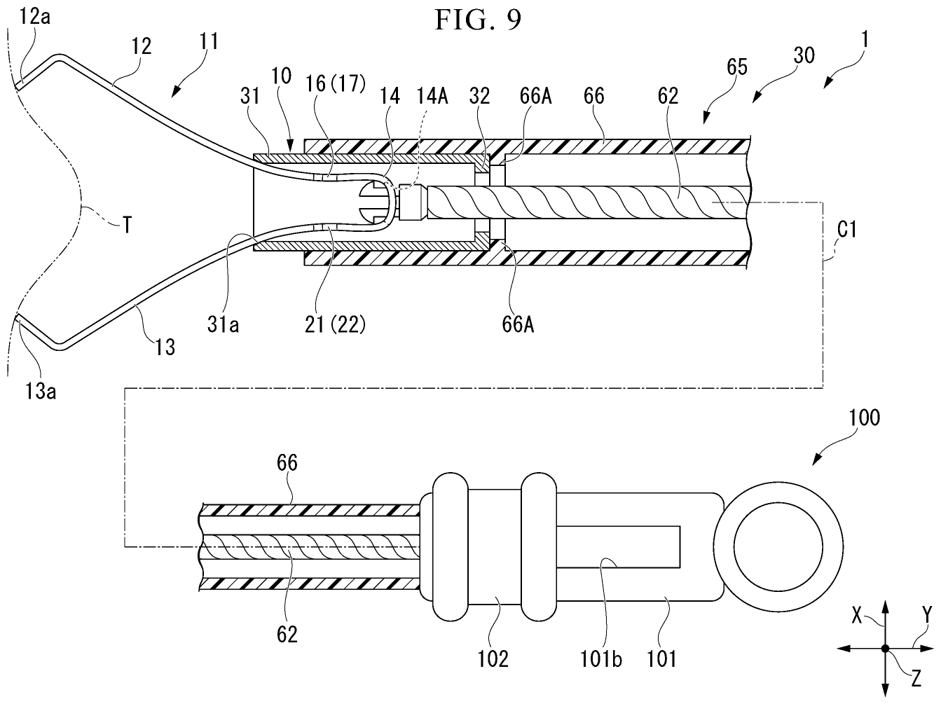

The medical device 1 according to the present embodiment includes a clip unit (treatment device) 10 provided on the distal end side and an applicator 30 (see FIG. 9). Hereinafter, for convenience of description, the clip unit 10 will be simply referred to as the clip 10 hereinafter. The medical device 1 according to the present embodiment is configured by attaching the clip 10 on a distal end portion of an applicator 30 described later.

Hereinafter, the configuration of the clip 10 according to the present embodiment will be described with reference to FIG. 1 to FIG. 3B.

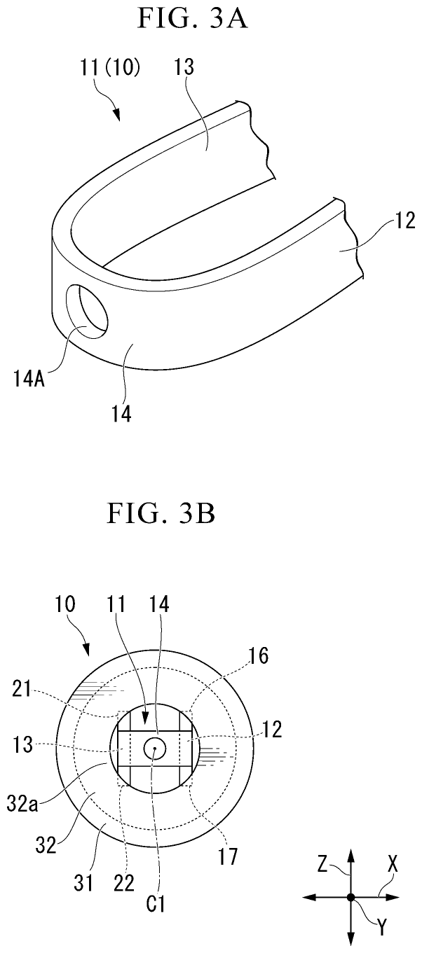

FIG. 1 is a partial cross-sectional planar view schematically showing the configuration of the clip 10 according to the present embodiment. FIG. 2 is a partial cross-sectional side view schematically showing the configuration of the clip unit according to the present embodiment. FIG. 3A is a perspective view schematically showing the configuration of the intermediate portion of the clip unit according to the present embodiment. FIG. 3B is a view of the clip unit according to the present embodiment as viewed from the proximal end side.

As shown in FIG. 1, the clip 10 according to the present embodiment includes an arm member 11 and a pressing tube 31. As shown in FIG. 1 and FIG. 2, the arm member 11 has a first arm 12, a second arm 13, and an intermediate portion 14.

### (Structure of Arm Member)

The first arm 12 and the second arm 13 extend from the proximal end side toward the distal end side and are arranged to face each other. As shown in FIG. 1, the first arm 12 and the second arm 13 may be formed at positions symmetrical with respect to the longitudinal axis of the pressing tube 31. According to the present embodiment, in a natural state, the first arm 12 and the second arm 13 are separated from each other, and have an elastic restoring force so as to make the distance between the first arm 12 and the second arm 13 to increase along the direction from the proximal end side toward the distal end side. In the present specification, the “natural state” means a state in which there is not any external force being applied to the arm member 11. A claw 12a extending toward the second arm 13 side is formed at the distal end portion of the first arm 12. A claw 13a extending toward the first arm 12 side is formed at the distal end portion of the second arm 13.

A pair of protrusions 16, 17 are formed on the first arm 12 of the arm member 11. More specifically, as shown in FIG. 2, the pair of protrusions 16, 17 project laterally orthogonal to the longitudinal direction in which the first arm 12 of the arm member 11 extends. The pair of protrusions 16, 17 may be formed at positions symmetrical with respect to the axis in the longitudinal direction in which the arm member 11 extends. Since the pair of protrusions 16, 17 are formed in the first arm 12, it is possible to lock the first arm 12 by a locking portion 32 formed on the proximal end side of the pressing tube 31, as will be described later. As shown in FIG. 3B, according to the present embodiment, in the pair of protrusions 16, 17 formed on the first arm 12, a distance from the tip in the protrusion direction of the protrusion 16 to the tip in the protrusion direction of the protrusion 17 in the axis Z direction maybe larger than a diameter of the opening formed in the locking portion 32.

Similarly, in the second arm 13 of the arm member 11, a pair of protrusions 21, 22 are formed. The pair of protrusions 21, 22 formed in the second arm 13 has the same configuration as the pair of protrusions 16, 17 formed in the first arm 12.

The intermediate portion 14 is located between the proximal end portion of the first arm 12 and the proximal end portion of the second arm 13. More specifically, as shown in FIG. 1 and FIG. 2, the intermediate portion 14 is positioned between the pair of protrusions 16, 17 on the first arm 12 of the arm member 11 and the pair of protrusions 21 and 22 on the second arm 13.

According to the present embodiment, as shown in FIG. 3A and FIG. 3B, an insertion port 14A for inserting an operation wire that will be described later is formed in a portion of the arm member 11 located on the most proximal end side of the intermediate portion 14. As shown in FIG. 3B, when viewed from the proximal end side along the longitudinal axis on which the arm member 11 extends, the insertion port 14A is formed in a circular shape.

According to the present embodiment, the insertion port 14A formed in the intermediate portion 14 is formed for inserting the distal end portion of the operation wire described later. An example that the insertion port 14A is formed in a circular shape so as to make it easy to insert the distal end of the elastically deformable operation wire is described; however, the present disclosure is not limited to this configuration. The shape of the insertion port 14A is not particularly limited as long as the distal end portion of the operation wire can be inserted and it is not easy for the distal end portion of the operation wire to be slipped from the insertion port 14A. Further, according to the present embodiment, in order to easily release the engagement between the operation wire 62 and the arm member 11 described later, the strength (rigidness) of the portion of the intermediate portion 14 of the arm member 11 where the insertion port 14A is formed may be set to be lower than other part of the intermediate portion 14. For example, in the intermediate portion 14 of the arm member 11, the thickness in the vicinity of the insertion port 14A may be formed thinner than the other portions.

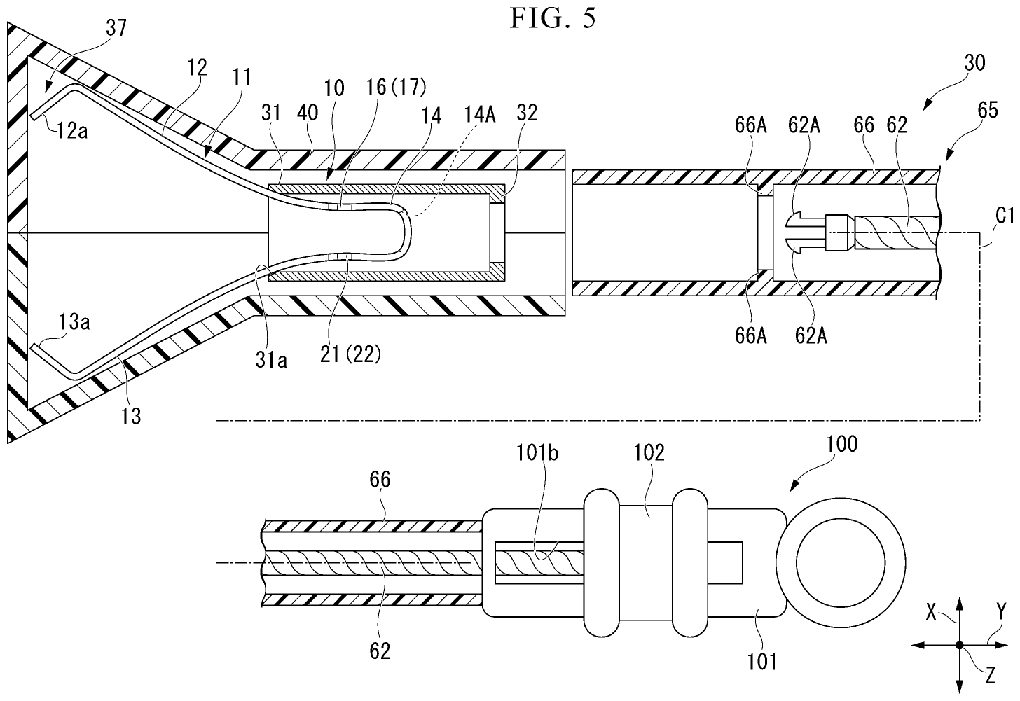

As shown in FIG. 1 and FIG. 2, the arm member 11 is formed to be in contact with a tapered surface 31a formed on the distal end side of the pressing tube 31 due to the elastic restoring force of the arm member 11 in the natural state in which there is not any external force being applied thereto. Here, the “natural state” means a state in which there is not any external force being applied to the arm member 11. According to the present embodiment, the elastic restoring force of the first arm 12 and the second arm 13 of the arm member 11 has an action of moving the arm member 11 itself toward the distal end side with respect to the pressing tube 31. Therefore, as shown in FIG. 1 and FIG. 2, the configuration of the arm member 11 on the proximal end side including the intermediate portion 14 and the insertion port 14A is located at a more distal end side than the locking portion 32, and is accommodated inside the pressing tube 31. Since the clip 10 including the arm member 11 is manufactured and then shipped in the state of being accommodated in the clip cartridge 40 described later, the arm member 11 does not unintentionally slip out from the pressing tube 31 (see FIG. 5).

Since the arm member 11 according to the present embodiment has the above-described configuration, as described later, when the arm member 11 retracts with respect to the pressing tube 31, the first arm 12 and the second arm 13 of the arm member 11 are in contact with the tapered surface 31a formed on the distal end side of the pressing tube 31, and the distance between the first arm 12 and the second arm 13 decreases. In other words, the first arm 12 and the second arm 13 can transition from the open configuration in which they are separated from each other to the closed configuration in which they approach each other. On the other hand, when the arm member 11 advances with respect to the pressing tube 31, the first arm 12 and the second arm 13 can transit from the closed configuration to the open configuration in which they are separated from each other.

In the arm member 11, the first arm 12 and the second arm 13 can be formed by bending a metal material such as a plate spring material formed from stainless steel. With this configuration, the arm member 11 can slide along the inner wall of the pressing tube 31 when moving inside the pressing tube 31 described below. For example, the arm member 11 is integrally formed by punching a plate material formed of a cobalt chrome alloy into a shape in which the first arm 12 and the second arm 13, the intermediate portion 14, and the pair of protrusions 16, 17 are expanded in a planar shape.

### (Structure of Pressing Tube)

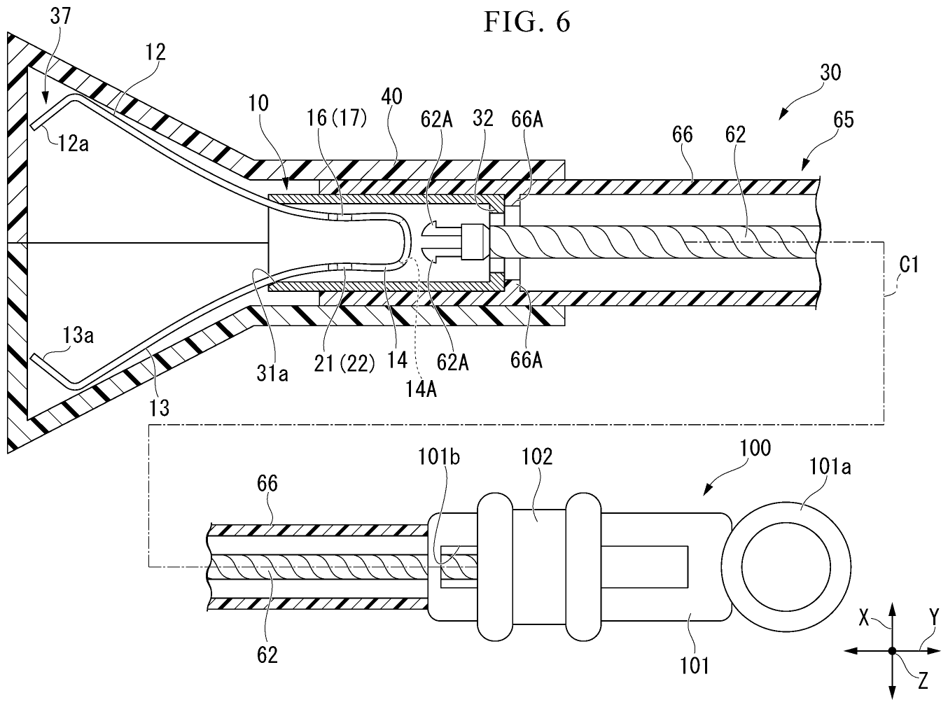

According to the present embodiment, the pressing tube 31 is formed as a pipe having a longitudinal axis and in a cylindrical shape. The pressing tube 31 has an inner diameter suitable for the intermediate portion 14 of the arm member 11 to enter (see FIG. 5). That is, the pressing tube 31 is formed with a lumen into which the first arm 12 and the second arm 13 of the arm member 11 are enterable. According to the present embodiment, the pressing tube 31 is formed such that the outer diameter thereof is smaller than the inner diameter of the sheath 66 of the insertion portion 65 described later (see FIG. 6).

These members including the arm member 11 that configure the clip 10 are made of a material such as a cobalt chromium alloy, titanium, stainless steel, or the like. The clip 10 is configured to be observable under MRI (Nuclear Magnetic Resonance Imaging).

As shown in FIG. 3, a tapered surface 31a is formed over the entire circumference on the inner wall of the distal end portion of the pressing tube 31. The tapered surface 31a has a diameter that increases toward the distal end side. According to the present embodiment, the pressing tube 31 may be integrally formed of a material such as 64 titanium alloy (Ti-6AL-4V) or cobalt chromium alloy.

### (Configuration of Applicator)

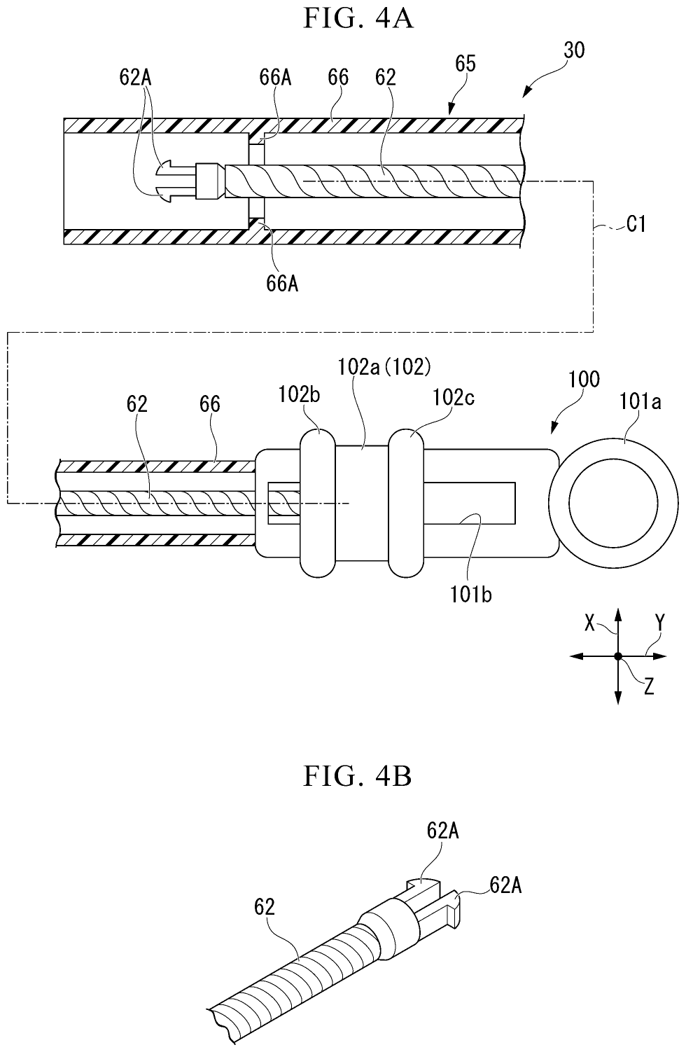

Next, the configuration of the applicator 30 according to the present embodiment will be described with reference to FIG. 4A and FIG. 4B. As shown in FIG. 4A, the applicator 30 according to the present embodiment is configured to have an insertion portion 65 and an operation portion 100.

The insertion portion 65 is located at more distal end side than the operation portion 100 and connected to the distal end of the operation portion 100. On the other hand, the operation portion 100 is attached to the proximal end portion of the insertion part 65.

### (Structure of Insertion Portion)

The insertion portion 65 of the applicator 30 includes a sheath 66 and an operation wire (wire) 62. The operation wire 62 is inserted into the sheath 66 to be advanceable and retractable in the sheath 66. The operation wire 62 is provided to transmit the force by the operator to operate the operation portion 100 (for example, the operation of pushing in the slider 102 and the operation of retracting the slider 102) to the clip 10.

The sheath 66 maybe a coil sheath formed of stainless steel such as SUS301 having high compress-resistance strength and extending along the longitudinal axis C1. In this case, the sheath 66 may be adopted as a coil formed by closely winding a wire (not shown) in the axial direction Y. The sheath 66 has flexibility and is strong against the compressive force in the longitudinal axis C1 direction.

According to the present embodiment, as shown in FIG. 4A, a protrusion 66A is formed on the inner circumferential surface of the sheath 66 to protrude radially inward of the sheath 66. In other words, in the sheath 66 in which the protrusion 66A is formed, a small-diameter portion having an inner diameter smaller than that of the other portion is formed. The inner diameter of the small diameter portion inside the sheath 66 in which the protrusion 66A is formed is smaller than the outer diameter of the pressing tube 31. The sheath 66 is configured in this manner such that the proximal end surface of the pressing tube 31 contacts (abuts) the protrusion 66A, and the pressing tube 31 can be received by the distal end portion of the sheath 66.

According to present embodiment, the pressing tube 31 only has to be received by the distal end portion of the sheath 66 by the pressing tube 31 and the protrusion 66A being engaged with each other, and the distance from the distal end surface of the sheath 66 to the protrusion 66A along the longitudinal axis of the sheath 66 may be suitably set. According to the present embodiment, the protrusion 66A may be formed over the entire circumference of the inner circumferential surface of the sheath 66, or may be formed at only a few places.

According to the present embodiment, the operation wire 62 is formed of, for example, a metal single wire or a twisted wire. As shown in FIG. 4B, the operation wire 62 is configured to have a pair of deformation portions 62A whose distal end portions are elastically deformable. The pair of deformation portions 62A are formed of a metal material similarly to the operation wire 62, and thus have durability that can be repeatedly used.

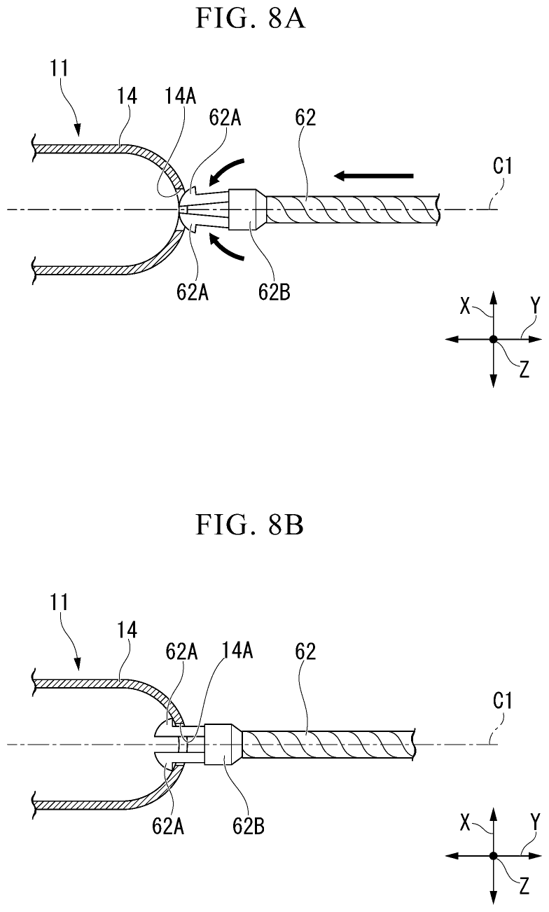

According to the present embodiment, the width of the distal end portion of the operation wire 62 in which the pair of deformation portions 62A is formed is larger than the width of the insertion port 14A formed in the intermediate portion of the arm member 11 described above. As shown in FIG. 8A and FIG. 8B, when the pair of deformation portions 62A are inserted into the insertion port 14A, the pair of deformation portions 62A are pressed inward in the radial direction while contacting the edges of the insertion port 14A. As a result, the pair of deformation portions 62A are elastically deformed and approach each other such that the width of the pair of deformation portions 62A becomes smaller and the pair of deformation portions 62A can pass through the insertion port 14A. When the pair of deformation portions 62A pass through the insertion port 14A and the engagement therebetween is released, the pair of deformation portions 62A restore to the initial shape due to the elastic restoring force. Accordingly, when the pair of deformation portions 62A pass through the insertion port 14A, they are engaged with each other as shown in FIG. 8B to restrict the movement of the operation wire 62 toward the proximal end side with respect to the arm member 11.

Further, a fixing portion 62B, which is a cylindrical member made of a metal material such as stainless steel, is provided on the distal end side of the operation wire 62. The width of the fixing portion 62B is equal to or larger than the width of the insertion port 14A in the intermediate portion 14 of the arm member 11. The operation wire 62 is fixed to the fixing portion 62B by various known methods such as bonding and welding. In the present embodiment, the operation wire 62, the fixing portion 62B, and the pair of deformation portions 62A are integrally formed. The pair of deformation portion 62A and fixing portion 62B can move forward and backward together with the operation wire 62 by the forward and backward movement of the operation wire 62.

### (Structure of Operation Portion)

As shown in FIG. 4A, the operation portion 100 includes an operation portion main body (handle) 101 and a slider 102.

The operation portion main body 101 is attached to the proximal end portion of the sheath 66. The operation portion main body 101 is formed in a rod shape extending in the axial direction Y, and has a finger hook portion 101a at the proximal end portion. The operation portion main body 101 is provided with a slit 101b extending in the axial direction Y.

The slider 102 is provided to be inserted into the operation portion main body 101. The slider 102 is slidable (advanceable and retractable) in the Y-axis direction with respect to the operation unit body 101. According to the present embodiment, when the slider 102 is advanced and retracted in the axial direction Y, the operation wire 62 and the pair of deformation portions 62A provided at the distal end of the operation wire 62 are advanced and retracted.

According to the present embodiment, in the state in which the operation wire 62 and the clip 10 are connected, the arm member 11 of the clip 10 advances or retracts together with the operation wire 62 by the advancement or retraction of the operation wire 62. As a result, the pair of first arm 12 and second arm 13 of the arm member 11 may be opened or closed.

The slider 102 is formed in a cylindrical shape. On the outer circumferential surface of the slider 102, a recess 102a is formed over the entire circumference. On the slider 102, a flange portion 102b, the recess 102a, and a flange portion 102c are formed in this order from the distal end side to the proximal end side in the axial direction Y. The pair of flange portion 102b and flange portion 102c have elliptical shapes when viewed in the axial direction Y. As a result, the slider 102 can be easily grasped, and space can be saved when the operation portion 100 is packed. The slider 102 engages with the slit 101b of the operation portion main body 101 to limit the movement range of the slider 102 in the axial direction Y with respect to the operation portion main body 101.

According to the present embodiment, the operation portion 100 may be adopted by using the configuration of the operation unit of various known endoscope treatment devices.

### (Operation to Attach the Clip to the Applicator)

Hereinafter, an operation of attaching the clip 10 according to the present embodiment to the applicator 30 will be described with reference to FIG. 5 to FIG. 8B.

As shown in FIG. 5, in the initial state, the clip 10 according to the present embodiment is accommodated in the clip cartridge 40. The clip cartridge 40 is configured to facilitate transportation and prevent contamination of the external environment during the process from the manufacture of the clip 10 until the clip 10 is actual used.

FIG. 5 is used to show only the positional relationship between the clip 10 and the clip cartridge 40 when the clip 10 is accommodated in the clip cartridge 40. The clip cartridge 40 according to the present embodiment is not limited to the configuration shown in FIG. 5. The clip cartridge 40 according to the present embodiment only has to be configured with the size suitably to accommodate the clip 10 and easy for the operator to take, and the shape thereof is not particularly limited. The clip cartridge 40 according to the present embodiment may employ the configuration of the clip case described in Japanese Unexamined Patent Application, First Publication No. 2009-011852, for example.

As shown in FIG. 5, in the clip cartridge 40, a clip accommodation portion (accommodation portion) 37 having an inner cavity capable of accommodating the clip 10 including the arm member 11 in the open configuration is formed. According to the present embodiment, the accommodation portion 37 only has to be able to accommodate the clip 10, and the shape and size are not particularly limited. For example, as shown in FIG. 5, the size of the inner cavity of the accommodation portion 37 in the radial direction of the clip cartridge 40 may be equal to or larger than the opening width between the first arm 12 and the second arm 13 of the arm member 11 in the open configuration.

The clip 10 is accommodated in the accommodation portion 37 along the longitudinal axis of the clip cartridge 40. As shown in FIG. 5, the first arm 12 and the second arm 13 of the arm member 11 of the clip 10 are in contact with the tapered surface formed on the distal end side of the pressing tube 31, and are in the open configuration in which the first arm 12 and the second arm 13 are separated from each other. In this state, the arm member 11 can relatively move to the distal end side with respect to the pressing tube 31 by the elastic restoring force of the first arm 12 and the second arm 13. As a result, as shown in FIG. 5, the arm member 11 is accommodated in the clip accommodating portion 37 of the clip cartridge 40 in a state where the intermediate portion 14 including the insertion port 14A is located inside the pressing tube 31.

The operator grasps the operation portion 100 and moves the applicator 30 including the sheath 66 toward the clip 10 so as to insert the applicator 30 into the clip cartridge 40. During this operation, the slider 102 is positioned at more proximal end side than the distal end surface of the slit 101b of the operation portion main body 101.

In the present embodiment, as described above, the pressing tube 31 can be received by the distal end portion of the sheath 66. Therefore, as shown in FIG. 6, the operator may insert the sheath 66 into the clip cartridge 40 until the proximal end surface of the pressing tube 31 contacts (abuts) the protrusion 66A formed on the inner circumferential surface of the sheath 66. In this state, the intermediate portion 14 of the arm member 11 of the clip 10 is located inside the pressing tube 31. The pair of deformation portions 62A provided on the distal end side of the operation wire 62 enter the pressing tube 31 at more distal end side with respect to the protrusion 66A formed on the inner circumferential surface of the sheath 66, and the pair of deformation portions 62A are positioned to be apart from the insertion port 14A in the intermediate portion 14 of the arm member 11 at the proximal end side thereof.

As shown in FIG. 6, according to the present embodiment, for convenience of description, an example in which the outer diameter of the pressing tube 31 is substantially equal to the inner diameter of a portion of the sheath 66 on the distal end side of the protrusion 66A is described; however, the configuration is not limited thereto. The pressing tube 31 only has to be received in a stable state without slipping from the distal end portion of the sheath 66, and a certain clearance may be formed between the outer circumferential surface of the pressing tube 31 and the inner circumferential surface of the sheath 66.

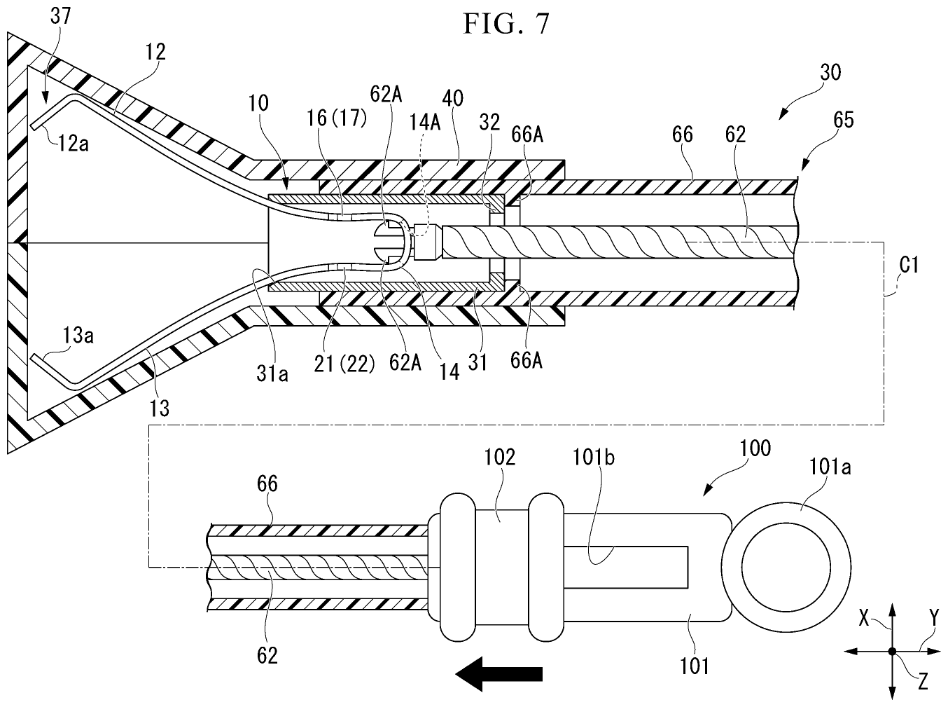

Thereafter, the operator pushes the slider 102 of the operation portion 100 toward the distal side while holding the slider 102. As shown in FIG. 7, when the operator pushes the slider 102 until the slider 102 comes in contact with the distal end surface of the slit 101b, the pair of deformation portions 62A provided at distal end of the operation wire 62 pass through the insertion port 14A formed in the intermediate portion 14 of the arm member 11. During this process, since the first arm 12 and the second arm 13 of the arm member 11 of the clip 10 are in contact with the distal end surface of the clip accommodation portion 37 of the clip cartridge 40, the position of the arm member 11 in the clip accommodation portion 37 is almost maintained. As shown in FIG. 7, according to the present embodiment, the operator pushes the slider 102 until the slider 102 comes into contact with the distal end surface of the slit 101b such that the pair of deformation portions 62A provided at the distal end of the operation wire 62 are engaged with the intermediate portion 14 of the arm member 11 and the operation wire 62 and the clip 10 are engaged with each other. The engagement portion of the operation wire 62 and the clip 10 is positioned inside the pressing tube 31 and at more proximal end side than the opening at the distal end side of the sheath 66. In this state, the operator may advance and retract the slider 102 so as to advance and retract the arm member 11 together with the operation wire 62.

More specifically, as shown in FIG. 8A and FIG. 8B, when the pair of deformation portions 62A provided at the distal end of the operation wire 62 come into contact with the insertion port 14A formed in the intermediate portion 14 of the arm member 11, since the width of the pair of deformation portions 62A is larger than the width of the insertion port 14A, the pair of deformation portions 62A are pressed into the insertion port 14A while the inclined surface formed in the outer circumference of the pair of deformation portions 62A are in contact with the edge of the insertion port 14A. As a result, the pair of deformation portions 62A may be elastically deformed and pass through the insertion port 14A in a reduced diameter state.

As shown in FIG. 8B, when the pair of deformation portions 62A pass through the insertion port 14A, the engagement between the pair of deformation portions 62A and the insertion port 14A is released. As a result, the pair of deformation portions 62A restore their original widths from the reduced-diameter state by the elastic restoring force of the pair of deformation portions 62A, and the pair of deformation portions 62A are locked to the insertion port 14A. In this state, even if the operator rotates the slider 102 around the longitudinal axis C1, the state in which the pair of deformation portions 62A are locked to the insertion port 14A is maintained.

As shown in FIG. 8B, according to the present embodiment, the fixing portion 62B provided at the proximal end side of the pair of deformation portions 62A is formed to have a width equal to or larger than the width of the insertion port 14A. Therefore, when the operator pushes the slider 102 such that the fixing portion 62B comes into contact with the edge of the insertion port 14A, the arm member 11 can be pushed toward the distal end side.

When the operator can confirm that the operation wire 62 is engaged with the arm member 11, the operator holds the operation portion 100 and removes the clip 10 and the operation wire 62 from the clip cartridge 40 as an integrated unit.

According to the above described operations, the operation of attaching the clip 10 according to the present embodiment on the applicator 30 are completed.

According to the present embodiment, when the operator removes the clip 10 from the clip cartridge 40 with the clip 10 attached to the applicator 30, the connection portion between the operation wire 62 and the arm member 11 is located at more distal end side than the opening at the proximal end side of the pressing tube 31 and inside the pressing tube 31 (see FIG. 7). The slider 102 in the operation portion 100 is in a position to come into contact with the distal end surface of the slit 101b along the slit 101b. That is, according to the present embodiment, when the slider 102 is moved to the most distal position, the connection portion between the operation wire 62 and the arm member 11 does not protrude from the pressing tube 31.

When the operator removes the clip 10 from the clip cartridge 40 with the clip 10 attached to the applicator 30, the connection portion between the operation wire 62 and the arm member 11 may be at more proximal end side than the opening at the proximal end side of the pressing tube 31. In this case, the connection portion between the operation wire 62 and the arm member 11 is located at the proximal end side than the opening at the distal end side of the sheath 66. That is, the connection portion between the operation wire 62 and the arm member 11 is located inside the sheath 66. The slider 102 in the operation portion 100 is in a position to come into contact with the distal end surface of the slit 101b along the slit 101b. That is, in this case, when the slider 102 is moved to the most distal position, the connection portion between the operation wire 62 and the arm member 11 does not protrude from the distal end opening of the sheath 66.

In either of the above-described cases, when the slider 102 is moved to the most distal position, the connection portion between the operation wire 62 and the arm member 11 is arranged inside the pressing tube 31 or inside the sheath 66.

The medical device 1 according to the present embodiment has the above-described configuration such that the operation of engaging the operation wire 62 and the clip 10 is performed at a position at more proximal end side than the distal end side opening of the sheath 66, that is, inside the sheath 66. As a result, it is possible to prevent the clip 10 from unintentionally slipping from the operation wire 62 by the connection portion between the operation wire and the clip 10 protruding from the distal end opening of the sheath 66.

### (Procedures by Medical Device)

Hereinafter, procedures for ligating the target tissue T using the medical device 1 according to the present embodiment having the above-described configuration will be described with reference to FIG. 9 to FIG. 13.

When the clip 10 is taken out from the clip cartridge 40 while the clip 10 being attached to the applicator 30, as shown in FIG. 9, the first arm 12 and the second arm 13 of the arm member 11 of the clip 10 are in the open configuration in which the first arm 12 and the second arm 13 are apart from each other while being contact with the tapered surface 31a formed at the distal end side of the pressing tube 31 due to the elastic restoring force of the first arm 12 and the second arm 13. In this state, the slider 102 is at the most advanced position in the slit 101b in the operation portion 100 at the proximal end side.

The elastic restoring force of the first arm 12 and the second arm 13 of the arm member 11 applies on the tapered surface 31a of the pressing tube 31 to cause the pressing tube 31 to move toward the proximal end side. Accordingly, as shown in FIG. 9, the proximal end surface of the pressing tube 31 is in contact with the distal end surface of the protrusion 66A of the sheath 66. In this state, the engagement portion (connection portion) between the operation wire 62 and the arm member 11 is located inside the pressing tube 31. The pair of protrusions 16, 17 formed on the first arm 12 of the arm member 11 and the pair of protrusions 21, 22 formed on the second arm 13 are located at the more distal end side than the locking portion 32 in the pressing tube 31.

The operator inserts an endoscope (not shown) into the body of the patient. Then, the operator inserts the medical device 1 from the proximal end portion of the channel of the endoscope, protrudes the medical device 1 from the distal end portion of the channel of the endoscope, and moves the medical device 1 to the vicinity of the target tissue T to be treated.

During the procedures, it is necessary to temporarily close the first arm 12 and the second arm 13 of the arm member 11 in the open configuration so that the medical device 1 can be smoothly inserted through the channel of the endoscope. In this case, for example, the operator may retract the slider 102 of the operation portion 100 at the proximal end side and pulls the first arm 12 and the second arm 13 into the pressing tube 31 so as to make the first arm 12 and the second arm 13 to be transitioned to a closed configuration in which the distance therebetween is approximately zero.

When the operator inserts the medical device 1 through the channel of the endoscope and moves it to the vicinity of the target tissue T, and then pushes the slider 102 of the operation portion 100 toward the distal end side, the distance between the first arm 12 and the second arm 13 increases and the arm member 11 is transitioned from the closed configuration to the open configuration.

At this time, the operator adjusts the opening width between the first arm 12 and the second arm 13 of the arm member 11 to an optimum value according to the size of the target tissue T by advancing and retracing the slider 102. As a result, as shown in FIG. 9, the operator may adjust the opening width between the first arm 12 and the second arm 13 of the arm member 11 to a value equal to or larger than the size of the target tissue T.

Next, the operator operates an endoscope (not shown), adjusts the orientation and posture of the arm member 11 of the clip 10, and presses the arm member 11 toward the target tissue T. By such an operation, the target tissue T is located between the first arm 12 and the second arm 13 of the arm member 11 in the open configuration. When the operator confirms that the target tissue T is located between the first arm 12 and the second arm 13, the operator may operate the endoscope to hold the target tissue by the first arm 12 and the second arm 13 of the arm member 11.

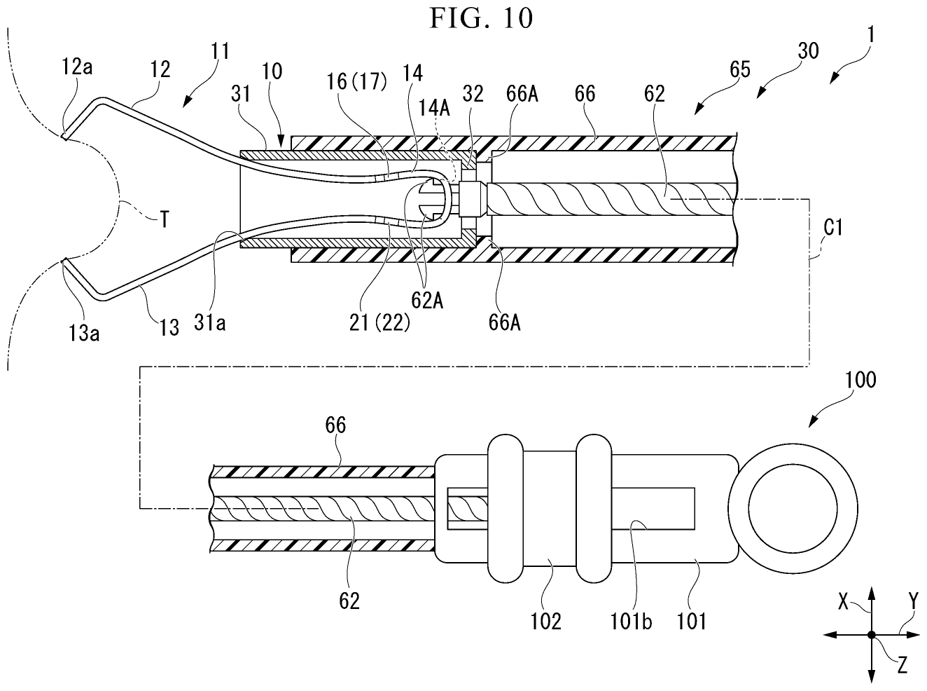

When the operator confirms that the target tissue T is located between the first arm 12 and the second arm 13, as shown in FIG. 10, the operator holds the operation portion main body 101 and retracts the slider 102 to the proximal end side. At this time, the operation wire 62 and the arm member 11 engaged with the operation wire 62 move together to the proximal end side. The first arm 12 and the second arm 13 are in contact with the tapered surface 31a provided at the distal end side of the pressing tube 31 while the first arm 12 elastically deforming toward the side of the second arm 13 and the second arm 13 elastically deforming toward the side of the first arm 12. As a result, the claw 12a provided on the distal end side of the first arm 12 and the claw 13A provided on the distal end side of the second arm 13 approach each other. In other words, the opening width of the arm member 11 between the first arm 12 and the second arm 13 is reduced, and the arm member 11 transitions from the open configuration to the closed configuration.

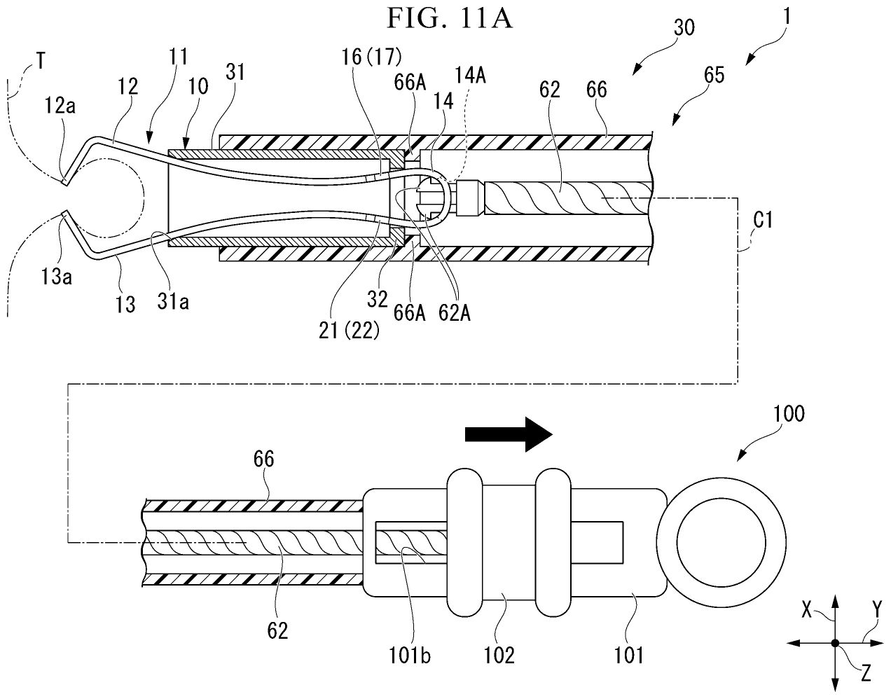

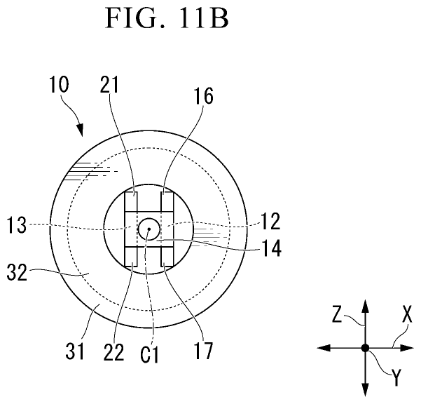

As a result of the operator retracting the slider 102 to the proximal end side, as shown in FIG. 11A, the target tissue T is held by the arm member 11 while the root of the target tissue T is tightly bound by the first arm 12 and the second arm 13. According to the present embodiment, the state in which the distance between the first arm 12 and the second arm 13 is substantially zero is also included in the closed configuration of the arm member 11. During the procedures, the connection portion between the operation wire 62 and the arm member 11 is moved to the proximal end side in the pressing tube 31.

When the operator retracts the slider 102 to the base end side, for example, as shown in FIG. 11A and FIG. 11B, the protrusions 16, 17 provided on the first arm 12 and the protrusions 21, 22 provided on the second arm 13 of the arm member 11 are in a state in which the protrusions 16, 17 and the protrusions 21, 22 are partially inside the locking portion 32. As shown in FIG. 11B, in this state, the intermediate portion 14 between the first arm 12 and the second arm 13 of the arm member 11 is compressed, and the distance between the proximal end portion of the first arm 12 and the proximal end portion of the second arm 13 is decreased. More specifically, as shown in FIG. 11B, when viewed from the proximal end side of the clip 10 along the direction of the longitudinal axis C1 of the pressing tube 31, the distance between the protrusion 16, 17 provided on the first arm 12 and the protrusions 21, 22 provided on the second arm 13 in the radial direction of the clip 10 becomes smaller than that in the state shown in FIG. 3B.

According to the present embodiment, in the procedures when the slider 102 is retracted to the proximal side until the protrusions 16, 17 of the first arm 12 and the protrusions 21, 22 of the second arm 13 of the arm member 11 completely pass through the locking portion 32, the operator may move the operation wire 62 and the clip 10 to the distal end side by pushing the slider 102 toward the distal end side. By this operation, the first arm 12 and the second arm 13 of the clip 10 may transition from the closed configuration to the open configuration again. That is, according to the medical device 1 according to the present embodiment, the operator only has to operate the slider 102 to perform an operation of grasping the target tissue T again using the clip 10. As described above, according to the medical device 1 according to the present embodiment, even if the slider 102 is pushed to the most distal position, it is impossible for the connection portion between the operation wire 62 and the clip 10 to protrude from the opening at the distal end side of the sheath 66 to be exposed to the outside. That is, it is possible to prevent the clip 10 from unintentionally slipping from the applicator 30.

When the operator can confirm that the target tissue T is grasped by the arm member 11 in a desired state, the operator further retracts the slider 102 to the proximal end side, and thereby the protrusions 16, 17 provided on the first arm 12 of the arm member 11 and the protrusions 21, 22 provided on the second arm 13 of the arm member 11 may move to the position at the proximal end side of the locking portion 32 by climbing over the locking portion 32 of the pressing tube 31.

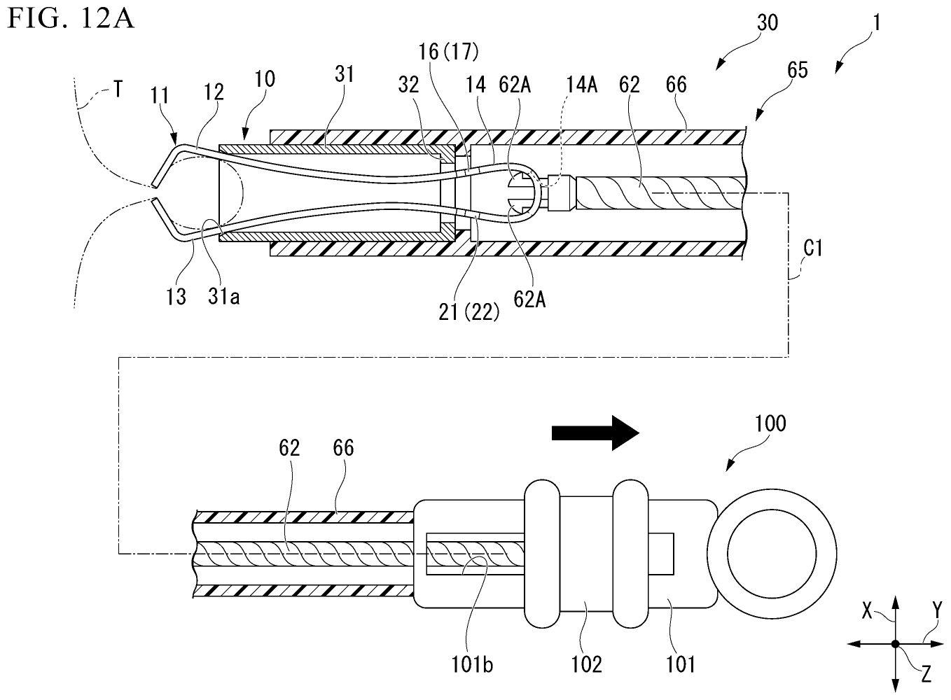

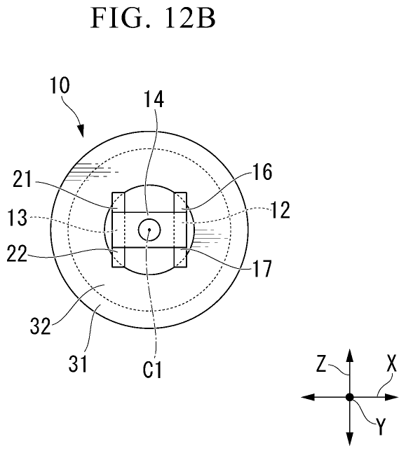

As shown in FIG. 12A and FIG. 12B, at this moment, the protrusions 16, 17 of the first arm 12 and the protrusions 21, 22 of the second arm 13 come into contact with the proximal end surface 32b of the locking portion 32 such that the first protrusion (first locked portion) 16 and the first protrusion (first locked portion) portion 17 are locked to the locking portion 32. The arm member 11 is in the closed configuration in which the distance between the first arm 12 and the second arm 13 is substantially zero. As shown in FIG. 12A, the root of the target tissue T is tightly bound by the first arm 12 and the second arm 13 of the arm member 11. In this state, the protrusions 16, 17 of the first arm 12 and the protrusions 21, 22 of the second arm 13 are locked to the locking portion 32 such that the advancement of the arm member 11 toward the distal end side with respect to the pressing tube 31 is restricted. As shown in FIG. 12B, due to the elastic restoring force of the intermediate portion 14 of the arm member 11 of the clip 10, when viewed from the proximal end side of the clip 10 along the direction of the longitudinal axis C1 of the pressing tube 31, the distance between the protrusions 16, 17 provided on the first arm 12 and the protrusions 21, 22 provided on the second arm 13 is substantially the same as that in the state shown in FIG. 3B.

In this state, the operator cannot operate the slider 102 to adjust the posture of the arm member 11 and re-grasp the target tissue T.

Subsequently, when the state in which the target tissue T is grasped by the arm member 11 in the closed state can be confirmed, the operator locks the state in which the target tissue T is grasped by the clip 10 and indwells the clip 10 in the body.

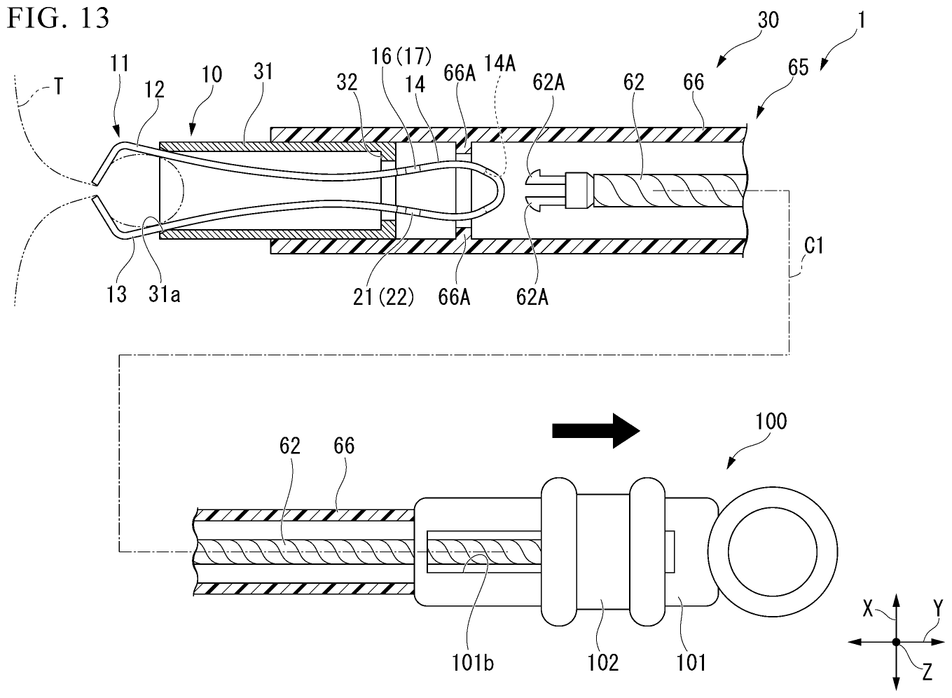

Specifically, when the operator further retracts the slider 102 toward the proximal end side, as shown in FIG. 13, the traction force applies on the connection portion between the operation wire 62 and the clip 10 located inside the sheath 66. As described above, according to the present embodiment, the strength of the intermediate portion 14 of the arm member 11 is set lower than that of the other portions. Accordingly, as shown in FIG. 13, when the traction force applying on the connection portion between the operation wire 62 and the clip 10 exceeds a predetermined value, the intermediate portion 14 of the arm member 11 is deformed and the insertion port 14A formed in the intermediate portion 14 expands.

As a result, the deformation portion 62A of the operation wire 62 is pulled out from the insertion port 14A and retracted into the sheath 66. Since the pair of deformation portions 62A provided at the distal end of the operation wire 62 are set to have high strength, it is possible to maintain a state of the deformation portion 62A in which there is almost no deformation or damage to the deformation portion 62A. On the other hand, the clip 10 configured from the arm member 11 and the pressing tube 31 is indwelled in the body while holding the target tissue T. In this case, the intermediate portion 14 of the arm member 11 is deformed; however, it is possible to prevent the broken pieces generated by the arm member 11 and the deformation portion 62A of the operation wire being broken from being unintentionally left in the body

After the clip 10 ligating the target tissue T is indwelled in the body, the operator operates the endoscope to take out the medical device 1 from the channel of the endoscope. Subsequently, the operator takes necessary measures and ends the series of procedures.

According to the medical device 1 according to the present embodiment, the operations of engaging the operation wire 62 and the clip 10 is performed inside the pressing tube 31 located at more proximal end side than the opening at the distal end side of the sheath 66 such that it is possible to prevent the clip 10 from unintentionally slipping from the operation wire 62.

According to the medical device 1 of the present embodiment, the operator may moves the operation wire 62 to the most distal position by pushing the slider 102 of the operation portion 100 on the proximal end side until the slider 102 comes in contact with the distal end surface of the slit 101b. Even if the operator pushes the slider 102 to the most distal position, the operation wire 62 does not project from the opening at the distal end side of the sheath 66. That is, in the procedures in which the operator operates the slider 102, it is not necessary for the operator to confirm the positional relationship between the operation wire 62 and the opening at the distal end side of the sheath 66, and the operation may be simplified.

### (Modification)

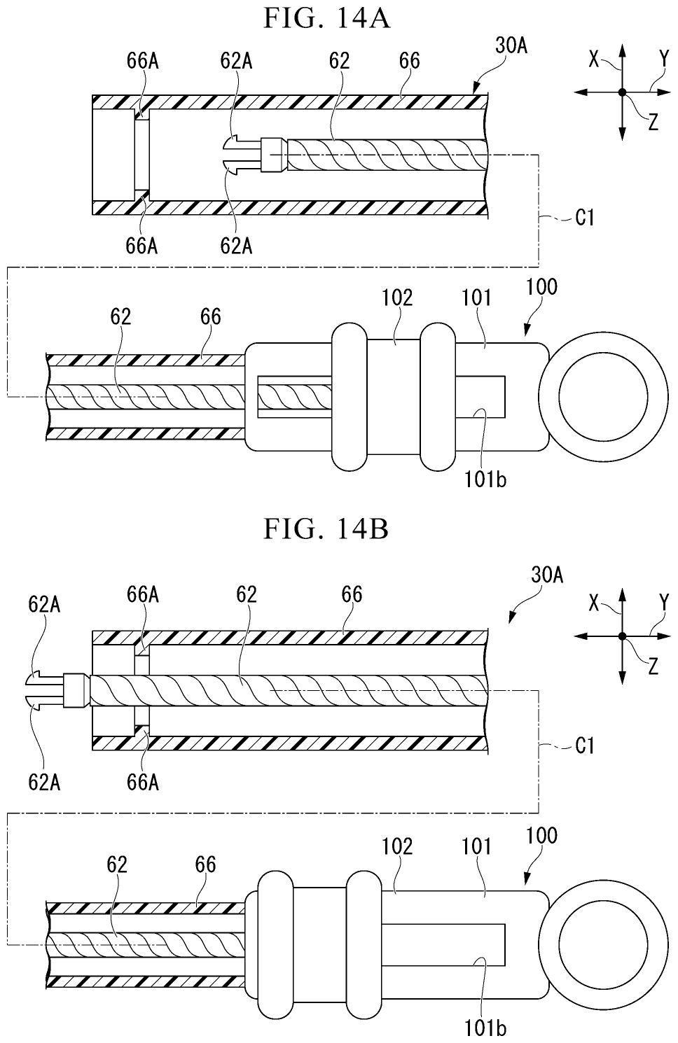

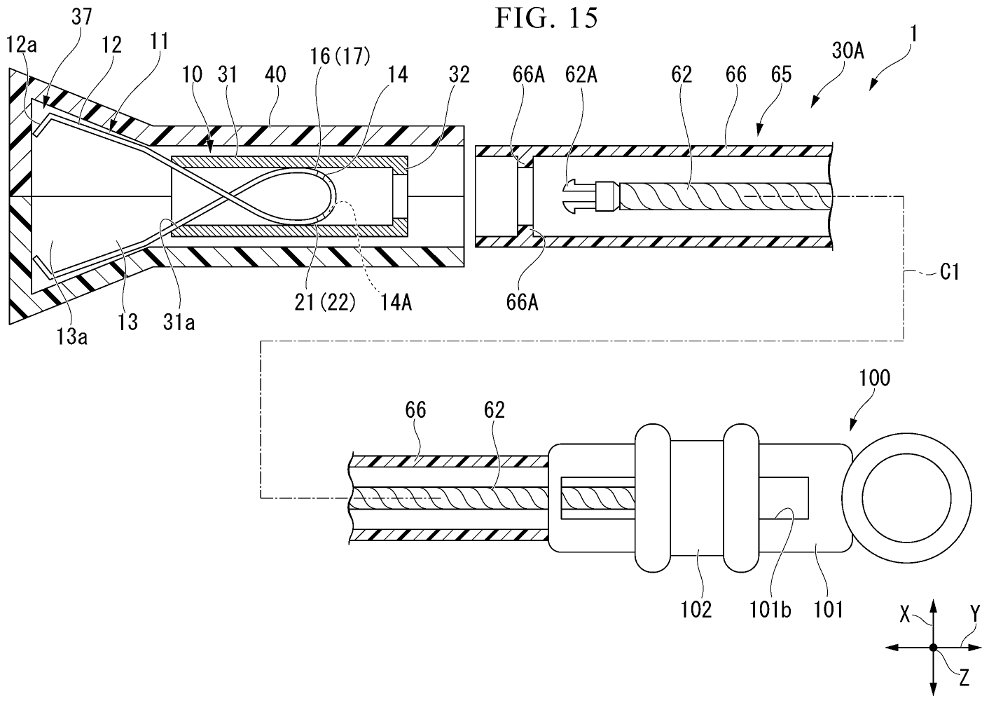

Hereinafter, a modification of the present embodiment will be described with reference to FIG. 14A to FIG. 17. FIG. 14A and FIG. 14B are views showing the configurations of the applicator 30A and the clip 10 according to the present modification. FIG. 15 to FIG. 17 are views showing an operation of attaching the clip 10 according to the present modification to the applicator 30A. The same configurations as those of the medical device 1 according to the above-described embodiment will be designated to the same reference numerals, the description thereof will be omitted, and points different from the above-described embodiment will be mainly described.

As shown in FIG. 14A and FIG. 14B, in the applicator 30A according to the present modification, when the operator advances the slider 102 of the operation portion 100 disposed at the proximal end side to the most distal position to come in contact with the distal end surface of the slit 101b, the distal end of the operation wire 62 and the pair of deformation portions 62A provided at the distal end of the operation wire 62 may protrude from the opening at the distal end side of the sheath 66. The protrusion 66A provided on the sheath 66 is formed in the vicinity of the opening on the distal end side of the sheath 66.

As shown in FIG. 15, in a state in which the clip 10 according to the present modification is arranged in the clip accommodation portion 37 of the clip cartridge 40, the intermediate portion 14 is positioned at more distal end side than the locking portion 32 and apart from the locking portion 32 inside the pressing tube 31. The first arm 12 and the second arm 13 of the arm member 11 are in the open configuration in which the first arm 12 and the second arm 13 are apart from each other.

### (Operation to Attach the Clip to the Applicator)

As shown in FIG. 15, when the clip 10 according to the present embodiment is attached to the applicator 30A, similarly to the above-described first embodiment, the operator holds the operation portion 100 and moves the applicator including the sheath 66 toward the clip 10 to insert the applicator 30A into the clip cartridge 40.

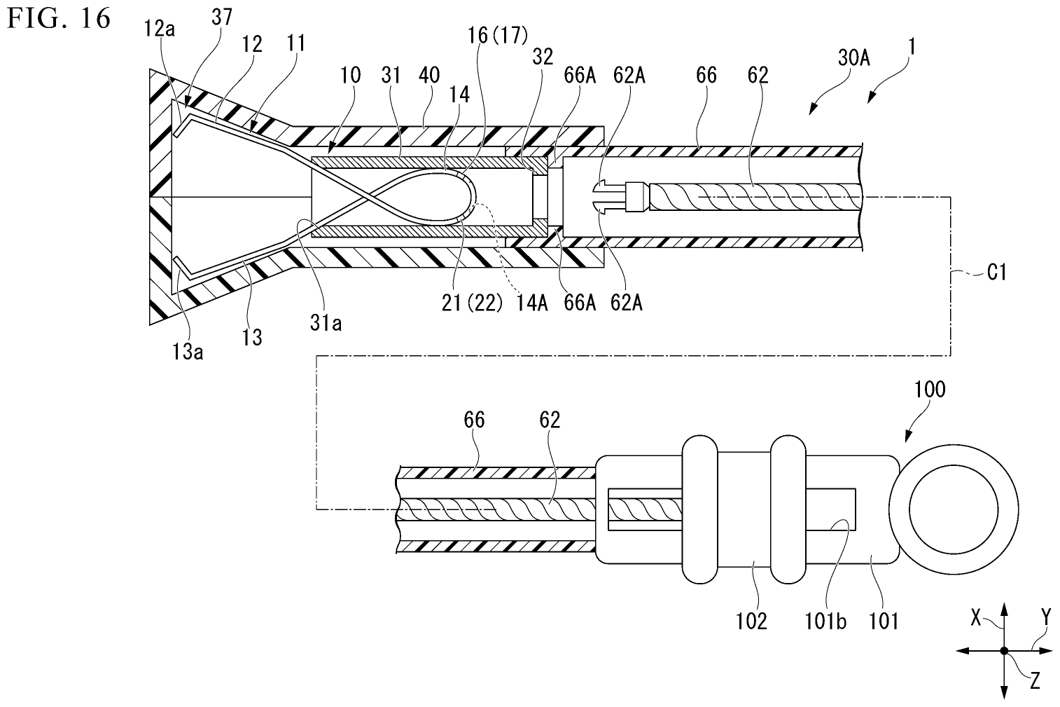

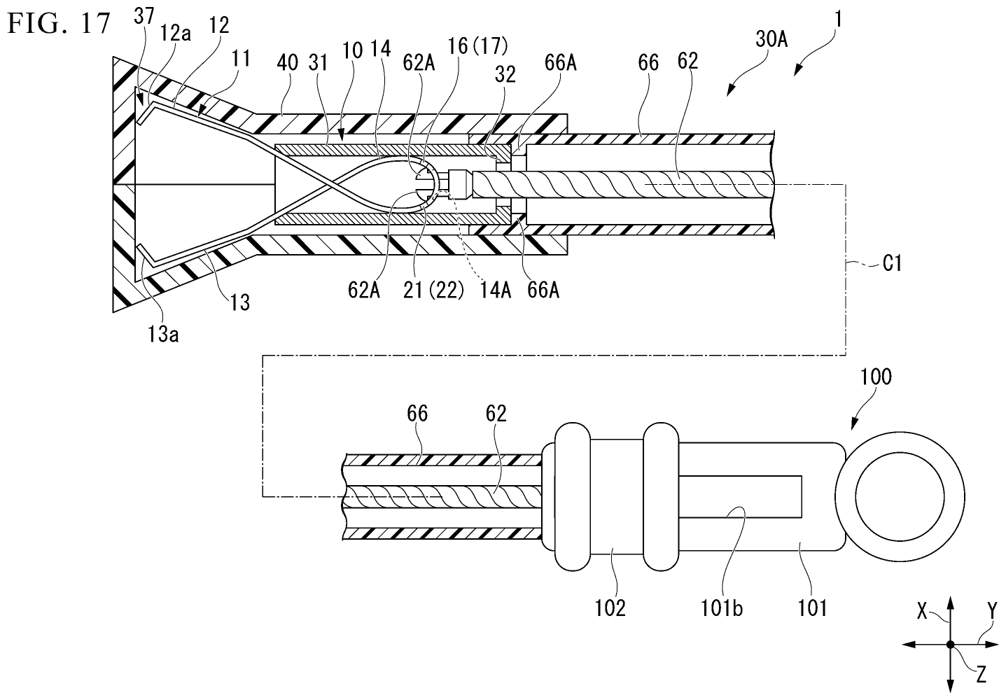

Subsequently, in a state in which the pressing tube 31 is supported by the protrusion 66A formed on the inner circumferential surface of the sheath 66, the operator grasps the slider 102 of the operation portion 100 and pushes the slider 102 toward the distal end side and toward the clip 10. As shown in FIG. 16 and FIG. 17, the operator may protrude the distal end of the operation wire 62 from the opening at the distal end side of the sheath 66 and insert the distal end of the operation wire 62 into the pressing tube 31 of the clip 10 by advancing the slider 102 to the position being in contact with the distal end surface of the slit 101b of the operation portion 100, that is, advancing the slider 102 to the most distal position. According to the operation, as shown in FIG. 17, the operator may insert the pair of deformation portions 62A provided at the distal end side of the operation wire 62 into the operation port 14A formed in the intermediate portion 14 of the arm member 11.

When the operator confirms that the operation wire 62 is engaged with the arm member 11, the operator holds the operation portion 100 and removes the clip 10 and the operation wire 62 as an integrated unit from the clip cartridge 40.

By the above-described operation, the operation of attaching the clip 10 according to the present embodiment to the applicator 30A is completed.

According to the present modification, in a state in which the pressing tube 31 is received by the distal end portion of the sheath 66, the operator may insert the pair of deformation portions 62A provided at the distal end side of the operation wire 62 into the operation port 14A formed in the intermediate portion 14 of the arm member 11 in the pressing tube 31 of the clip 10 by advancing the slider 102 of the operation portion 100 to the most distal position. According to the present modification, as shown in FIG. 17, the engagement portion (connection portion) between the operation wire 62 and the clip 10 is positioned at more distal end side than the opening at the distal end side of the sheath 66; however, the engagement portion is accommodated in the pressing tube 31 such that similarly to the above-described first embodiment, it is possible to prevent the operation wire 62 of the clip 10 from being unintentionally slipped.

### Second Embodiment

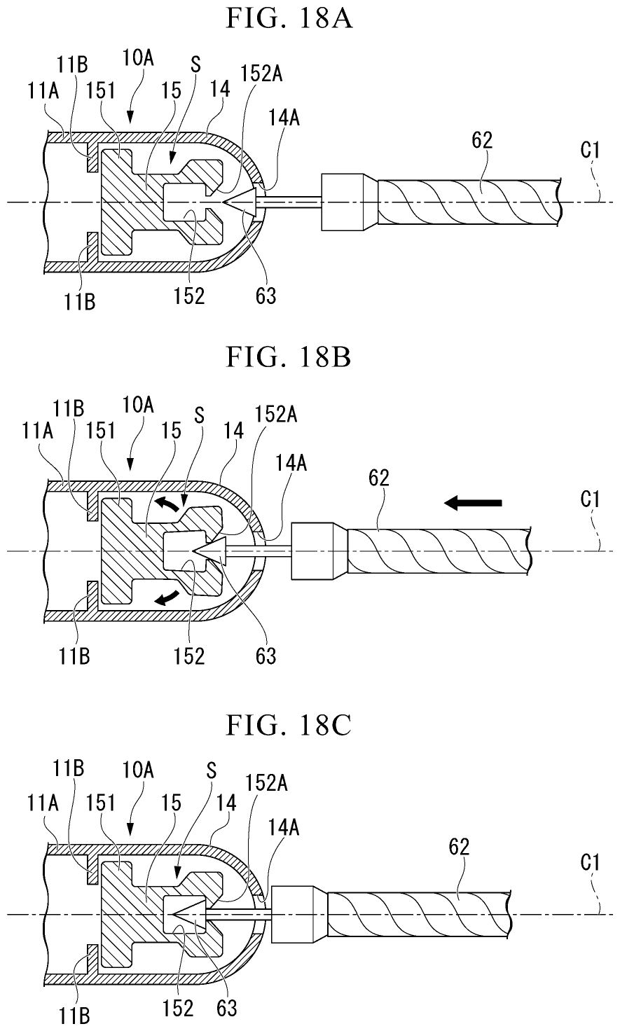

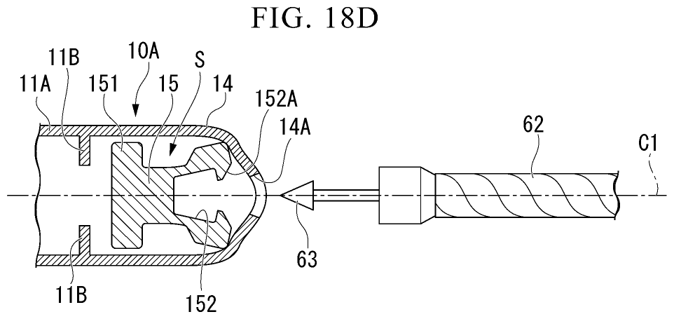

Hereinafter, the configuration of a medical device 2 according to a second embodiment of the present disclosure will be described with reference to FIG. 18A to FIG. 18D. Hereinafter, the same configurations as those of the medical device 1 according to the first embodiment described above will be designated to the same references, the description thereof will be omitted, and differences from the above embodiment will be mainly described.

As shown in FIG. 18A to FIG. 18D, the medical device 2 according to the present embodiment is different from the above-described first embodiment in the configuration of the connection portion formed by engaging the operation wire 62 and the clip 10A. Accordingly, this point will be mainly described in the following description.

As shown in FIG. 18A, on the proximal end side of the arm member 11A of the clip 10A of the medical device 2 according to the present embodiment, a space S surrounded by a pair of walls 11B that are formed to project inward in the radial direction from the arm member 11A and the intermediate portion 14 is formed. A block member 15 is accommodated in the space S without being fixed to the arm member 11A.

The block member 15 has a main body 151 and a notch 152, and for example, the block member 15 is formed by molding a material such as a biocompatible resin that is elastically deformable. According to the present embodiment, the block member 15 is accommodated in the space S in a state in which it is substantially immovable with respect to the arm member 11A in the direction of the axis C1 and the radial direction of the arm member 11A. The block member 15 only has to be accommodated in the space S in the state in which the block member 15 is substantially immovable with respect to the arm member 11A, and the shape thereof is not particularly limited. For example, the main body 151 of the block member 15 may have a width substantially equal to the inner diameter of the arm member 11A at the position where the pair of walls 11B are formed. The main body 151 may have a length being at least equal to a distance from the pair of walls 11B to the insertion port 14A along the direction of the axis C1. The notch 152 is formed with an opening portion 152A that opens toward the proximal end side in the direction of the axis C1. The opening portion 152A of the notch 152 has a size such that a hook (convex portion) 63 having an arrowhead shape formed on the distal end portion of the operation wire 62 can enter (see FIG. 18C).

According to the present embodiment, the block member 15 having the above-described configuration is accommodated in the space S formed at the proximal end side of the arm member 11A in a state in which the block member 15 is substantially impossible to rotate around the direction of the axis C1. In other words, the opening portion 152A of the block member 15 is maintained in a state to open toward the insertion port 14A at the proximal end side of the arm member 11A.

The operation wire 62 according to the present embodiment has a hook (convex portion) 63 having an arrowhead shape on the distal end side thereof. According to the present embodiment, since the hook 63 provided at the disposed at the distal end of the operation wire 62 has to be repeatedly used, it is preferable that the hook 63 is formed of a metal material having a predetermined strength, similar to that of the operation wire 62.

According to the present embodiment, with regard to the hook 63 provided at the distal end of the operation wire 62, the width of the hook 63 in the radial direction of the operation wire 62 may be smaller than the width of the insertion port 14A formed in the intermediate portion 14 of the arm member 11A, and larger than the width of the entrance of the notch 152 formed in the block member 15.

### (Operation to Attach the Clip to the Applicator)

The operator moves the hook 63 together with the operation wire 62 toward the block member 15 by pushing the slider of the operation unit (not shown) toward the distal end side. As shown in FIG. 18A and FIG. 18B, when the operator pushes the slider, the hook 63 passes through the insertion port 14A, enters the space S in the arm member 11A, and then enters the opening portion 152A formed at the proximal end side of the block member 15. At this time, since the width of the hook 63 in the radial direction is larger than the width of the opening 152A of the notch 152 of the block member 15, the block member 15 is elastically deformed at the opening portion 152A.

As shown in FIG. 18B, when the opening portion 152A of the notch 152 of the block member 15 is elastically deformed, the hook 63 enters the opening portion 152A of the block member 15 to be pressed by the notch 152 by the operator pushing the slider of the operation portion toward the distal end side. Accordingly, as shown in FIG. 18C, the hook 63 and the block member 15 are engaged with each other. According to the present embodiment, since the block member 15 is accommodated in the space S in the state in which the block member 15 is substantially immoveable with respect to the arm member 11A, if the hook 63 and the block member 15 are engaged with each other, it can be considered that the hook 63 is connected to the arm member 11A. In this state, since the arm member 11A may advance and retract when the operator advances and retracts the operation wire 62, the arm member 11A can be opened or closed.

According to the present embodiment, as shown in FIG. 18A, in the state before the hook 63 connected to the distal end side of the operation wire 62 is inserted into the insertion port 14A of the arm member 11, the width of the hook 63 in the radial direction of the arm member 11A is smaller than the width of the insertion port 14A. On the other hand, as shown in FIG. 18C, the hook 63 enters the space S from the insertion port 14A of the arm member 11 and the hook 63 is inserted into the opening portion 152A of the block member 15 to be engaged with the notch 152 of the block member 15 such that the configuration provided by engaging the hook 63 and the block member 15 has a width larger than the width of the insertion port 14A in the radial direction of the arm member 11A.

According to the present embodiment, the combination of the hook 63 and the block member 15 can be considered as a link mechanism for engaging the operation wire 62 and the arm member 11. As shown in FIG. 18A, a small-width configuration of the link mechanism is defined as the state in which the width of the hook 63 is smaller than the width of the insertion port 14A. As shown in FIG. 18C, a large-width configuration of the link mechanism is defined as the state in which the width of the configuration formed by engaging the hook 63 and the block member 15 is larger than the width of the insertion port 14A.

According to the present embodiment, the link mechanism transitions from the small-width configuration to the large-width configuration by the hook 63 entering the space S through the insertion port 14A and engaging with the notch 152 of the block member 15. The link mechanism is configured to cause the operation wire 62 and the arm member 11A to transition from the release configuration in which the engagement between the operation wire 62 and the arm member 11A is released to the engagement configuration in which they are engaged with each other.

According to the present embodiment, as shown in FIG. 18C, similarly to the first embodiment, the operator uses the medical device 2 configured by connecting the operation wire 62 and the arm member 11A to treat (ligate) the target tissue inside the body.

When the operator finishes the treatment using the medical device 2 according to the present embodiment and indwells the clip 10 in the body, for example, by the operation of retracting the slider of the operation unit (not shown) at the proximal end side, it is possible to release the connection state between the hook 63 and the arm member 11A. More specifically, the operator may retract the operation wire 62 and the hook 63 to the proximal end side by retracting the slider of the operation unit (not shown) to the proximal end side. At this time, since the hook 63 and the block member 15 are engaged with each other, the block member 15 is retracted to the proximal end side together with the hook 63 and the block member 15 comes in contact with the intermediate portion 14 of the arm member 11A inside the space S. In this state, when the operator operates a slider (not shown) to further retract the operation wire 62, as shown in FIG. 18D, for example, due to the traction force for retracting the operation wire 62 and the hook 63 by the operator, the portion in the vicinity of the opening portion 152A of the block member 15 and the insertion port 14A of the arm member 11 is plastically deformed such that it is possible to remove the operation wire 62 and the hook 63. In this state, the opening portion 152A of the block member 15 and the insertion port 14A of the arm member 11 are in a state of being expanded in diameter. However, the block member 15 is maintained to be accommodated in the space S formed at the proximal end side of the arm member 11 and the block member 15 is not removed from the insertion port 14A.

According to the medical device 2 according to the present embodiment, the hook 63 provided at the distal end of the operation wire 62 is inserted into the insertion port 14A formed in the intermediate portion 14 of the arm member 11, and the hook 63 is engaged with the notch 152 of the block member 15 accommodated in the space S at the proximal end side of the arm member 11 such that the operation wire 62 and the clip 10 can be transitioned from the released configuration to the engagement configuration.

According to the medical device 2 according to the present embodiment, the operation of engaging the operation wire 62 and the clip 10 is performed in the space S at the proximal end side of the arm member 11. Accordingly, the connection portion between the operation wire 62 and the clip 10 is not exposed to the outside of the sheath or the pressing tube (not shown), and it is possible to prevent the clip 10 from being unintentionally slipped from the operation wire 62.

### Third Embodiment

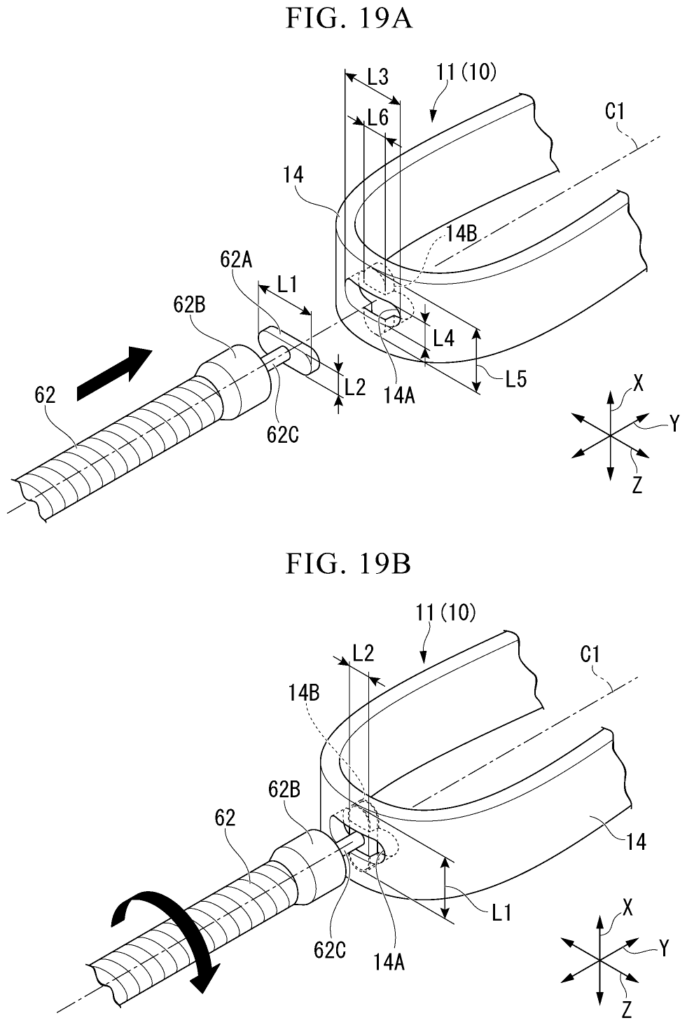

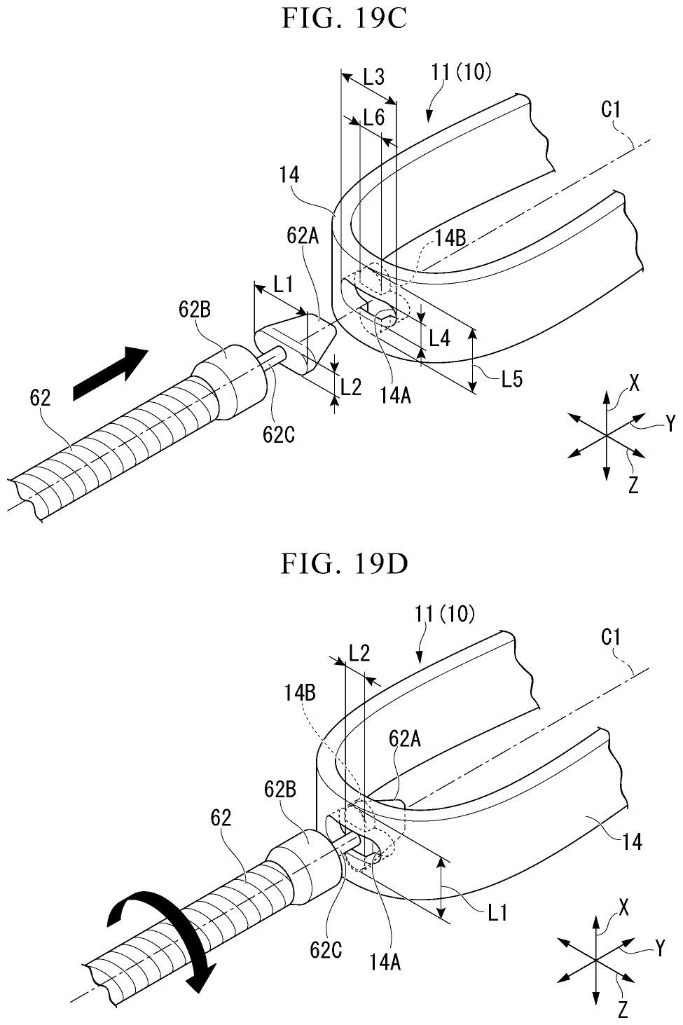

The configuration of a medical device 3 according to a third embodiment of the present disclosure will be described with reference to FIG. 19A to FIG. 25. Hereinafter, the same configurations as those of the medical device 1 according to the first embodiment described above will be designated by the same reference numerals, the description thereof will be omitted, and differences from the above embodiment will be mainly described.

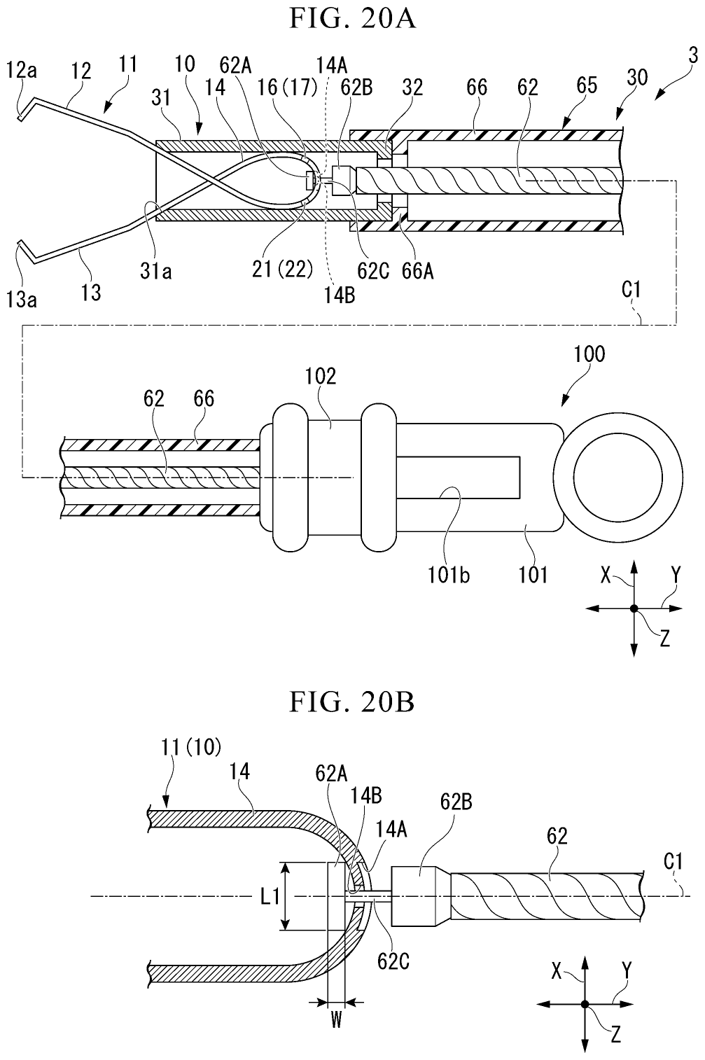

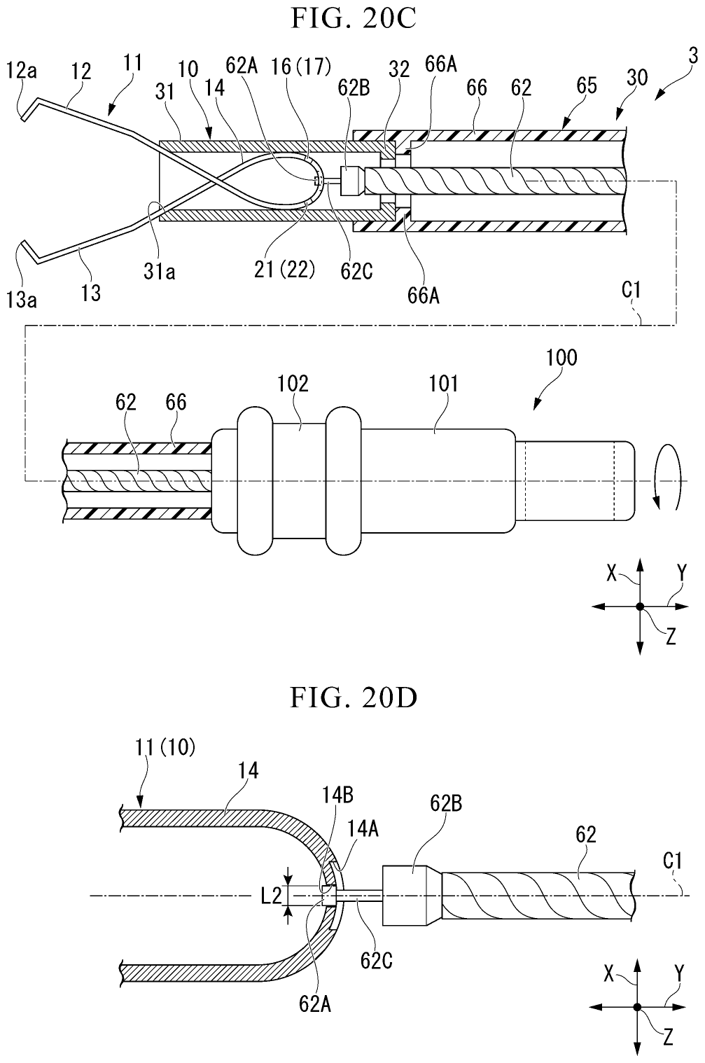

FIG. 19A and FIG. 19B are views showing a configuration of a connection portion where the arm member 11 and the operation wire 62 according to the present embodiment are engaged with each other. FIG. 19C and FIG. 19D are views showing a configuration of a connection portion where the arm member 11 and the operation wire 62 according to a modification of the present embodiment are engaged with each other. FIG. 20A to FIG. 25 are views showing procedures of attaching the clip 10 to the applicator 30 in the medical device 3 according to the present embodiment and treating the target tissues.

According to the present embodiment, the engagement structure in which the clip 10 and the operation wire 62 are engaged with each other is different from that in each of the above-described embodiments. Accordingly, the present embodiment will be described focusing on this point.

### (Structure of Connection Portion Between Arm Member and Operation Wire)

According to the present embodiment, a fixing portion 62B, a plate-shaped hook 62A, and a shaft portion 62C connecting the hook 62A and the fixing portion 62B are formed at the distal end side of the operation wire 62. As shown in FIG. 19A, the hook 62A is formed to have a thickness W in the longitudinal axis direction of the operation wire 62. The hook 62A has a first width L1 in a first direction around the longitudinal axis along which the operation wire 62 extends and a second width L2 in a second direction different from the first direction around the longitudinal axis. According to the present embodiment, for convenience of explanation, in the orthogonal coordinate system shown in FIG. 19A, the Y-axis direction is defined as the longitudinal axis direction of the operation wire 62 (the direction of the axis C1), the Z-axis direction is defined as the first direction, and the X-axis direction is defined as the second direction. That is, the hook 62A has a first width L1 in the Z-axis direction, a second width L2 in the X-axis direction, and a thickness W in the Y-axis direction.

An insertion port 14b penetrating the intermediate portion 14 is formed in the intermediate portion 14 of the arm member 11. As shown in FIG. 19A, the insertion port 14A has a third width L3 in the Z-axis direction and a fourth width L4 in the X-axis direction. In the insertion port 14A, for example, the third width L3 is set to be larger than the first width L1 of the hook 62A, and the fourth width L4 is set to be smaller than the first width L1 of the hook 62A and larger than the second width L2 of the hook 62A.

In other words, according to the present embodiment, as shown in FIG. 19A, only when the direction in which the first width L1 of the hook 62A is defined and the direction in which the third width L3 of the insertion port 14A is defined match, the hook 62A can be inserted into the insertion port 14A.

In the intermediate portion 14, an engagement groove 14B extending from the inside to the outside of the arm member 11 along a direction different from the direction in which the insertion port 14A extends is formed. For convenience of description, as shown in FIG. 19A and FIG. 19B, an example in which the engagement groove 14B has a fifth width L5 in the X-axis direction, a sixth width L6 in the Z-axis direction, and a depth W1 in the Y-axis direction will be described. However, the configuration according to the present disclosure is not limited thereto. According to the present embodiment, the hook 62A only has to be able to fit in the engagement groove 14B by inserting the hook 62A into the insertion port 14A and rotating around the longitudinal axis direction of the operation wire 62 by a predetermined angle, and the arrangement and dimensions of the engagement groove 14 are not particularly limited. For example, in the engagement groove 14B, the fifth width L5 may be set to be larger than the first width L1 described above, and the sixth width L6 may be set to be smaller than the first width L1 described above and larger than the second width L2.

The engagement groove 14B only has to have a depth suitable for engaging the hook 62A and the arm member 11 by fitting at least part of the hook 62A thereinto. For example, the engagement groove 14B only has to be formed with the depth W1 that is set to be smaller than the thickness of the portion in the vicinity of the insertion port 14A of the intermediate portion of the arm member 11, and the depth W1 may be set to be larger or smaller than the thickness W of the hook 62A in the Y-axis direction.

According to the present embodiment, the fixing portion 62B has an outer diameter set to be larger than the fourth width L4 of the insertion port 14A, and the fixing portion 62B is formed in a substantially cylindrical shape. The fixing portion 62B may press the intermediate portion 14 of the arm member 11 and move the intermediate portion 14 toward the distal end side in a state in which the hook 62A is engaged with the engagement groove 14B and the operation wire 62 and the clip 10 are connected. The fixing portion 62B is a member configured to transmit the force for moving the operation wire 62 toward the distal end side by the operation of the operator to push the slider (not shown) of the operation portion at the proximal end side.

However, according to the present embodiment, the elastic restoring force of the first arm 12 and the second arm 13 of the arm member 11 may be appropriately set so as to make the arm member 11 to be in contact with the inner circumferential surface of the pressing pipe (not shown) and advances or retracts the arm member 11. In other words, according to the present embodiment, the fixing portion 62B is not an essential component.

In FIG. 19A and FIG. 19B, the example in which the hook 62A of the operation wire 62 according to the present embodiment is formed in a plate shape is described; however, the configuration according to the present disclosure it not limited thereto. For example, the hook 62A of the operation wire 62 according to the present embodiment may be formed in a rounded substantially triangular shape as shown in FIG. 19C and FIG. 19D. In this case, the hook 62A of the operation wire 62 may be also engaged with the engagement groove 14B by rotating the hook 62A by a predetermined angle after being inserted into the insertion port 14A.

The hook 62A of the operation wire 62 according to the present embodiment is formed in the shape shown in FIG. 19C and FIG. 19D such that when the operation wire 62 is inserted into the insertion port 14A, there is no necessity to adjust the orientation of the hook 62A to align with the direction in which the insertion port 14A is formed in advance. More specifically, for example, when the operation wire 62 is pushed into the insertion port 14A, even if the direction in which the first width L1 of the hook 62A is formed and the Z-axis direction form an angle from zero degree to 90 degrees (equal to or larger than zero degree and equal to or smaller than 90 degrees), the hook 62A is rotated in the circumferential direction of the operation wire 62 while the outer circumferential surface of the hook 62A comes in contact with the edge of the insertion port 14A. As a result, when the operation wire 62 is inserted into the insertion port 14A, the hook 62A can be rotated in the direction in which the insertion port 14A is formed only by applying the force to push the operation wire 62 in the Y-axis direction.

### (Operation to Attach Clip to Applicator)

Hereinafter, with reference from FIG. 20A to FIG. 25, in the medical device 3 according to the present embodiment, an operation of attaching the clip 10 to the applicator 30 and procedures of using the medical device 3 according to the present embodiment will be described.

The operator holds the operation portion 100 on the proximal end side and moves the sheath 66 and the operation wire 62 integrally to the distal end side. The operator moves the slider 102 of the operation portion 100 at the proximal end side to the distal end side in a state where the proximal end surface of the pressing tube 31 is in contact with the protrusion 66A formed on the inner circumferential surface of the sheath 66. As shown in FIG. 20A, when the slider 102 comes into contact with the distal end surface of the slit 101b of the operation portion main body 101, that is, when the slider 102 moves to the most advanced position, the hook 62A provided on the distal end side of the operation wire 62 may be inserted into the insertion port 14A formed in the intermediate portion 14 of the arm member 11. At this time, as shown in FIG. 20B, the hook 62A provided at the distal end side of the operation wire 62 passes through the insertion port 14A, and inside the arm member 11, the hook 62A is locked to the inner circumferential surface of the intermediate portion 14 of the arm member 11. As shown in FIG. 20B, the direction in which the first width L1 of the hook 62A is defined intersects (be orthogonal to) with the direction in which the engagement groove 14B of the intermediate portion 14 of the arm member 11 extends such that it is impossible for the hook 62A to enter the engagement groove 14B and fit into the engagement groove 14B. Hereinafter, for convenience of description, the direction in which the first width L1 of the hook 62A is defined is referred to as the direction in which the hook 62A extends.

According to the present embodiment, in the operation shown in FIG. 20A, the case in which the extending direction of the hook 62A provided on the distal end side of the operation wire 62 and the extending direction of the insertion port 14A coincide with each other has been described; however, the configuration of the present disclosure is not limited thereto. In a case in which the direction in which the hook 62A extends and the direction in which the insertion port 14A extends intersect with each other, for example, the operator may push the slider 102 toward the distal end side to make the hook 62A to come in contact with the intermediate portion 14 of the arm member 11, and then operate (for example, rotate) the operation portion 100 so as to adjust the extending direction of the hook 62A and insert the hook 62A into the insertion port 14A.

Subsequently, the operator engages the hook 62b with the engaging groove 14B formed at the inner side of the intermediate portion 14 of the arm member 11 by grasping and operating the operation portion 100 provided at the proximal end side. As shown in FIG. 20C, according to the present embodiment, since the respective extending directions of the insertion port 14A and the engagement groove 14B in the intermediate portion 14 are orthogonal to each other, for example, the operator may operate the operating portion 100 to rotate the operation wire 62 by 90 degrees. As a result, as shown in FIG. 20D, the hook 62A can be engaged with the engagement groove 14B formed at the inner side of the intermediate portion 14 of the arm member 11.

When the hook 62A of the operation wire 62 is engaged with the engagement groove 14B formed at the inner side of the intermediate portion 14 of the arm member 11, the operation by the operator with respect to the slider 102 of the operation portion 100 is transmitted to both of the operation wire 62 and the arm member 11. In other words, the arm member 11 may be moved or rotated together with the operation wire 62 by the operation of pushing/pulling back the slider 102 by the operator, a rotating operation, or the like. Accordingly, the operator may advance/retract the arm member 11 and adjust the posture of the arm member 11 by operating the slider 102 of the operation portion 100.

According to the above-described operations, the operation of attaching the clip 10 according to the present embodiment to the applicator 30 is completed.

In the present embodiment, in the state in which the clip 10 is attached to the applicator 30, as shown in FIG. 20A to FIG. 20D, at a position where the slider 102 comes in contact with the distal end surface of the slit 101b of the operation portion main body 101 of the operation portion 100, the pressing tube 31 is received by the distal end portion of the sheath 66, and the connection portion between the operation wire 62 and the arm member 11 is located at more distal end side than the opening at the distal end side of the sheath 66 and accommodated in the pressing tube 31. In other words, according to the present embodiment, similarly to each of the above-described embodiments, it is possible to prevent the connection portion between the operation wire 62 and the arm member 11 from being exposed to the outside, and it is possible to prevent the clip 10 from being unintentionally slipping from the operation wire 62.

According to the present embodiment, when the width of the hook 62A in the X-axis direction is referred to as a reference, as shown in FIG. 19A, before the hook 62A is inserted into the insertion port 14A, the width in the X-axis direction is the second width L2. On the other hand, as shown in FIG. 19B, when the hook 62A is inserted into the insertion port 14A and then rotated by 90 degrees to be engaged with the engagement groove 14B, the width of the hook 62A in the X-axis direction is the first width L1 that is larger than the second width L2. That is, according to the present embodiment, the hook 62A is rotated in the circumferential direction around the longitudinal axis of the operation wire 62 to transition from the small-width configuration shown in FIG. 19A to the large-width configuration shown in Fig.19B. As a result, the operation wire 62 and the arm member 11 may transition from the release configuration in which the operation wire 62 and the arm member 11 is not engaged with each other to the engaged configuration in which they are engaged with each other.

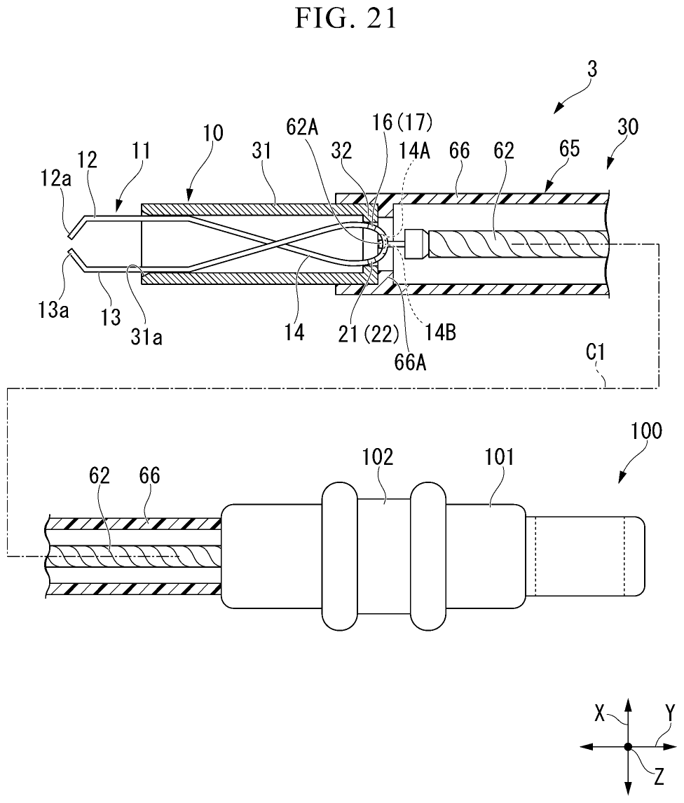

In the state in which the operation wire 62 and the arm member 11 are engaged with each other to configure the medical device 3, the operator may treat the target tissue with the medical device 3 according to the operations shown in FIG. 21 to FIG. 25.

As shown in FIG. 21, in order to smoothly insert the medical device 3 through the channel of the endoscope (not shown), the operator may retract the slider of the operation portion 100 at the proximal end side similarly to the operations of each of the above-described embodiments so as to once close the first arm 12 and the second arm 13 of the arm member 11 in the open configuration.

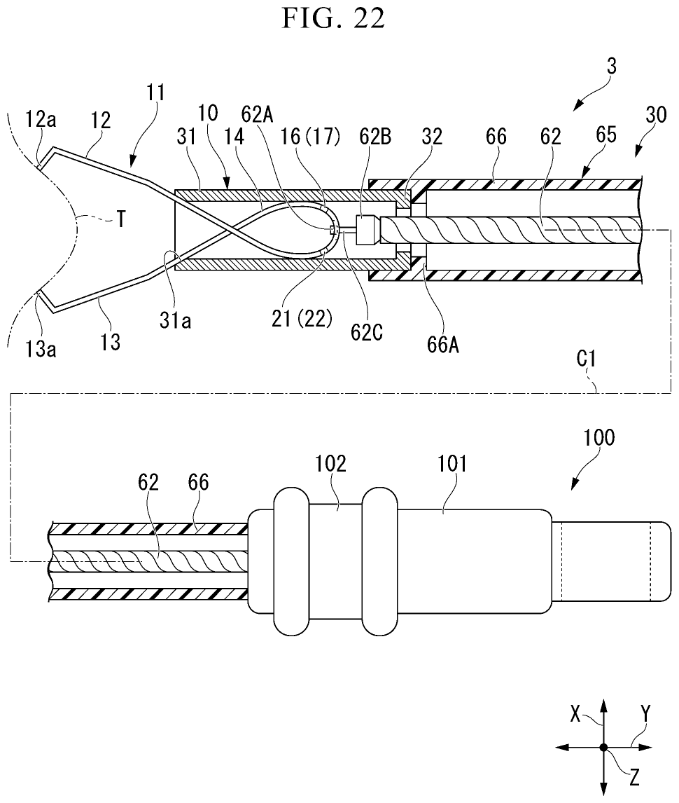

When the operator inserts the medical device 3 through the channel of the endoscope and moves the medical device 3 to the vicinity of the target tissue T, and then pushes the slider 102 of the operation portion 100 toward the distal end side, the arm member 11 may transition from the closed configuration to the open configuration such that the distance between the first arm 12 and the second arm 13 increases. As shown in FIG. 22, even the slider 102 is moved to the position where the slider 102 comes in contact with the distal end surface of the slit 101b of the main body 101 of the operation portion 100, the connection portion between the operation wire 62 and the arm member 11 is positioned inside the pressing tube 31. Therefore, according to the medical device 3 of the present embodiment, similarly to the above-described embodiments, during the process that the first arm 12 and the second arm 13 of the arm member 11 transition between the open configuration and the closed configuration by the operations to the slider 102, the connection portion between the operation wire 62 and the arm member 11 is not exposed to the outside. That is, according to the medical device 3 according to the present embodiment, it is possible to prevent the clip 10 from unintentionally slipping from the operation wire 62.

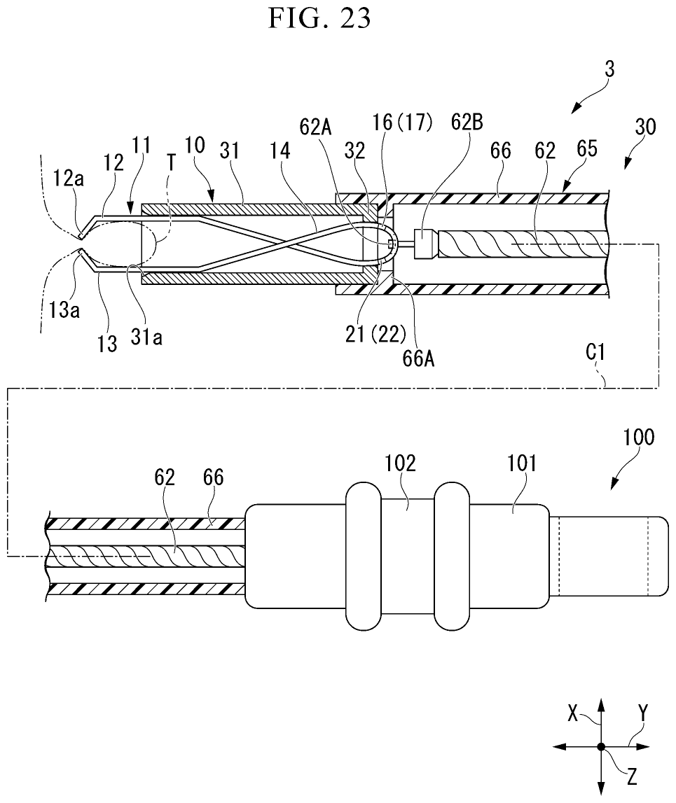

Subsequently, the operator may treat (ligate) the target tissue T by using the medical device 3 according to the present embodiment similarly to the above-described embodiments. When the operator confirms that the target tissue T is grasped by the arm member 11 in a desired state, the operator retracts the slider 102 of the operation portion 100 to the proximal end side as shown in FIG. 23 such that the protrusions 16, 17 of the first arm 12 and the protrusions 21, 22 of the second arm 13 to come in contact with the proximal end surface 32b of the locking portion 32, and the first locked portion 16 and the first locked portion 17 are locked to the locking portion 32. In this state, the connection portion between the operation wire 62 and the clip 10 is located inside the sheath 66 at more proximal end side than the opening at the proximal end side of the pressing tube 31.

According to such operations, the clip 10 is locked in the state of grasping the target tissue T, and the operator cannot re-grasp the target tissue T by operating the slider 102.

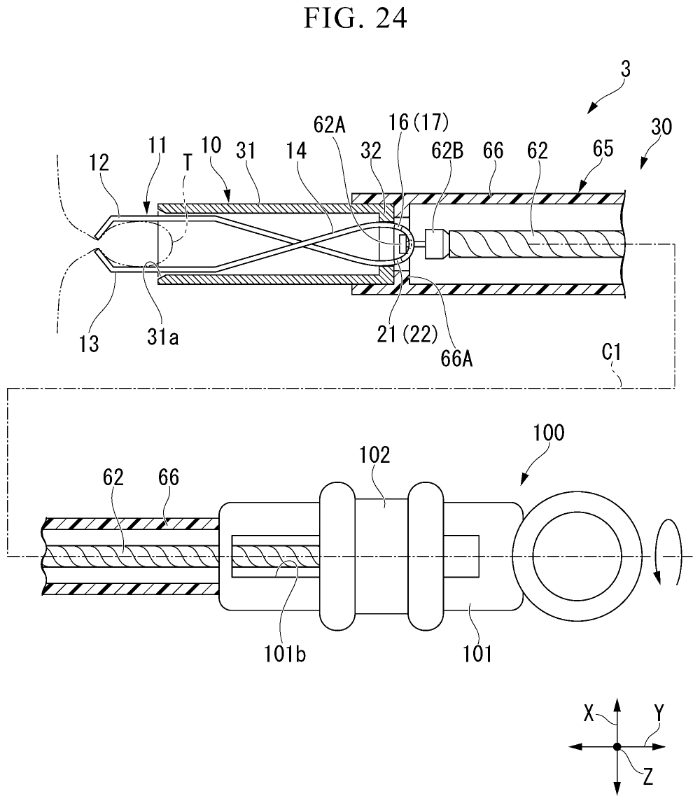

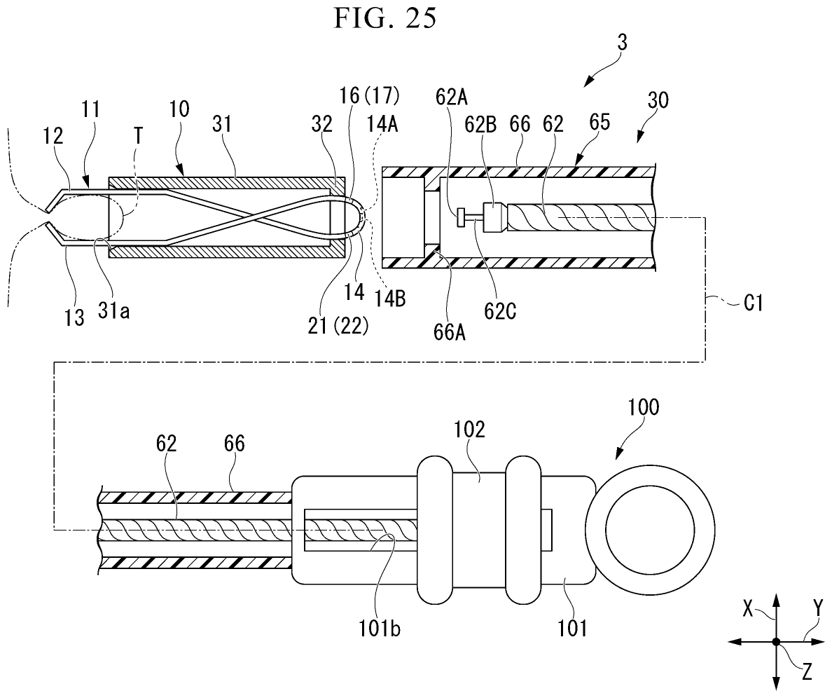

When the operator confirms that the state in which the target tissue T is grasped by the clip 10 is locked, the operator releases the engagement between the clip 10 and the operation wire 62 by the operation shown in FIG. 24 and FIG. 25 to indwell the clip 10 in the body.

Specifically, as shown in FIG. 24, when the operator slightly pushes the slider 102 of the operation portion 100 at the proximal end side, since the arm member 11 is locked by the locking portion 32 of the pressing tube 31, the hook 62A at the distal end of the operation wire 62 may be protruded from the engagement groove 14B. Then, in this state, for example, the operator may operate the operation portion 100 and rotate the operation wire 62 by 90 degrees so that the extending direction of the hook 62A and the extending direction of the insertion port 14A may be coincided with each other.

As shown in FIG. 25, when the direction in which the hook 62A at the distal end of the operation wire 62 extends and the direction in which the insertion port 14A extends are coincided with each other, the operator may retract the slider 102 to the proximal end side such that the hook 62A passes through the insertion port 14A, separated from the arm member 11 of the clip 10 and retracted into the sheath 66. Accordingly, the engagement between the operation wire 62 and the clip 10 is released.

After the clip 10 ligating the target tissue T is indwelled in the body, the operator may operate the endoscope to take out the medical device 3 from the channel of the endoscope. Subsequently, the operator may take necessary measures and ends the series of procedures.

According to the medical device 3 according to the present embodiment, after inserting the hook 62A in the direction aligned with the insertion port 14A formed in the intermediate portion 14 of the arm member 11 and then rotating the operation wire 62 in the circumferential direction around the longitudinal axis, it is possible to engage the hook 62A with the engagement groove 14B formed in the intermediate portion 14 so as to make the operation wire 62 and the clip 10 to transition from the release configuration to the engagement configuration.

According to the medical device 3 according to the present embodiment, the operation of engaging the operation wire 62 and the clip 10 is performed inside the pressing tube 31 such that the connection portion between the operation wire 62 and the clip 10 is not exposed to the outside, and it is possible to prevent the clip 10 from unintentionally slipping from the operation wire 62.

According to the medical device 3 according to the present embodiment, in the operation of releasing the engagement between the operation wire 62 and the clip 10, there is not necessity to retract the slider 102 with a large amount of force and break the intermediate portion 14 of the arm member 11. Accordingly, the procedures may be performed more easily. Since neither the arm member 11 nor the operation wire 62 is broken, it is possible to prevent contamination from occurring inside the body of the patient.

### Fourth Embodiment

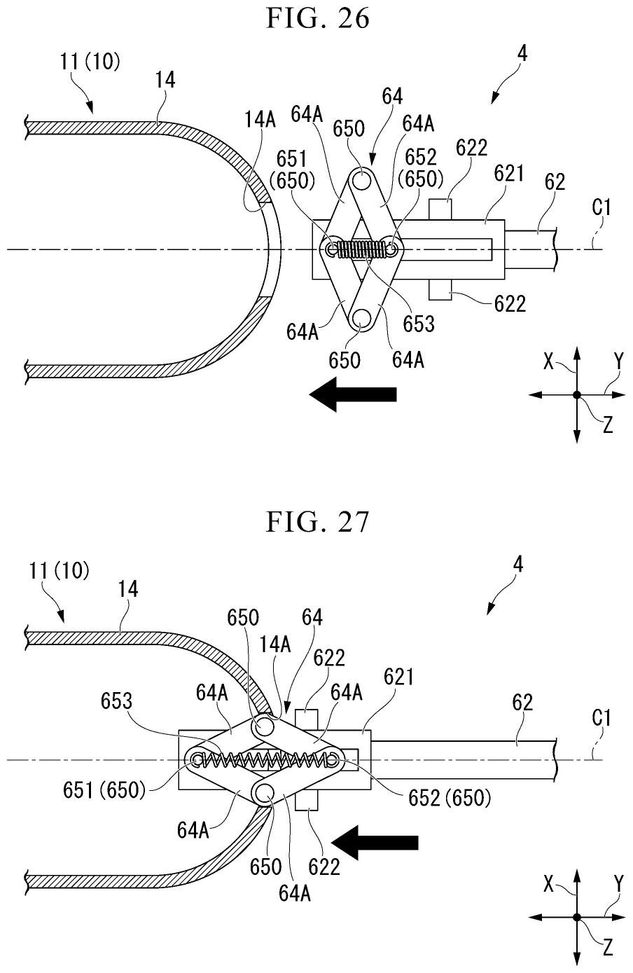

Hereinafter, a medical device 4 according to a fourth embodiment of the present disclosure will be described with reference to FIG. 26 to FIG. 28. Hereinafter, the same configurations as those of the medical device 1 according to the first embodiment described above will be designated by the same reference numerals, the description thereof will be omitted, and differences from the above embodiment will be mainly described.

As shown in FIG. 26, the medical device 4 according to the present embodiment is provided with a link mechanism 64 configured by four arms 64A connected in a rhombic shape on the distal end side of the operation wire 62.

As shown in FIG. 26, the link mechanism 64 is configured from four arms 64A that are connected in a rhombic shape. The two adjacent arms 64A in the link mechanism 64 are connected by a pin 650 provided therebetween, and each of the arms 64 can rotate about the pin 650 as a rotation center. The pin 651 that connects the two arms 64A located at the distal end side and the pin 652 that connects the two arms 64A located at the proximal end side are connected by an elastic member (spring) 653. As shown in FIG. 26, in the link mechanism 64, the pin 651 located at the distal end side is fixed to the fixing portion 621 of the operation wire 62, and the pin 652 located at the proximal end side is movable along a groove formed in the fixing portion 621 of the operation wire 62.

As shown in FIG. 26, in the fixing portion 621 of the operation wire 62, a pair of protrusions 622 protruding outward in the radial direction of the operation wire 62 are formed. According to the present embodiment, the width of the fixing portion 621 of the operation wire 62 where the pair of protrusions 622 are formed may be larger than the width of the insertion port 14A formed in the intermediate portion 14 of the arm member 11. According to the present embodiment, similarly to the fixing portion 62B described in the above-described third embodiment, the pair of protrusions 622 are configured to move the arm member 11 toward the distal end side when the link mechanism 64 is engaged with the insertion port 14A of the arm member 11.

Similar to the above-described third embodiment, the pair of protrusions 622 are not essential elements.

### (Operation to Attach Clip to Applicator)

When the clip 10 according to the present embodiment is attached to the applicator 30, the operator pushes the slider of the operation unit at the proximal end side (not shown) toward the proximal end side to move the operation wire 62 and the link mechanism 64 toward the arm member 11. According to the present embodiment, the elastic member 653 of the link mechanism 64 is biased in the direction of drawing the pin 651 and the pin 652. Accordingly, as shown in FIG. 26, in the process before the link mechanism 64 comes in contact with the insertion port 14A formed in the intermediate portion 14 of the arm member 11, the link mechanism 64 is formed in the rhombic shape that the width in the radial direction of the operation wire 6 is larger than the width of the operation wire 62 in the longitudinal direction. In the link mechanism 64, the width of the operation wire 62 in the radial direction is larger than the width of the insertion port 14A.

As shown in FIG. 27, when two arms 64A provided at the distal end side of the link mechanism 64 come into contact with the insertion port 14A, the force by the operator to push the slider is transmitted to the operation wire 62 and the link mechanism 64, and the two arms 64A press against the edge of the insertion port 14A. On the other hand, the reaction force from the pressed insertion port 14A is applied to the two arms 64A on the distal end side such that the elastic member 653 is extended and the pin 652 at the proximal end side is moved to the proximal end side. As a result, the width of the operation wire 62 in the radial direction is decreased and the width of the operation wire 62 in the axis direction is increased.

As shown in FIG. 27, when the width of the operation wire 62 of the link mechanism 64 in the radial direction is reduced to be equal to or smaller than the width of the insertion port 14A, the link mechanism 64 may pass through the insertion port 14A and enter the inside of the arm member 11. According to the present embodiment, in the state in which the link mechanism 64 partially enters the insertion port 14A, by the operation of pushing the slider by the operator, the link mechanism 64 is moved toward the distal end side, and due to the elastic restoring force of the elastic member 653, the width of the operation wire 62 in the radial direction is increased, and the width of the operation wire 62 in the axial direction is decreased.

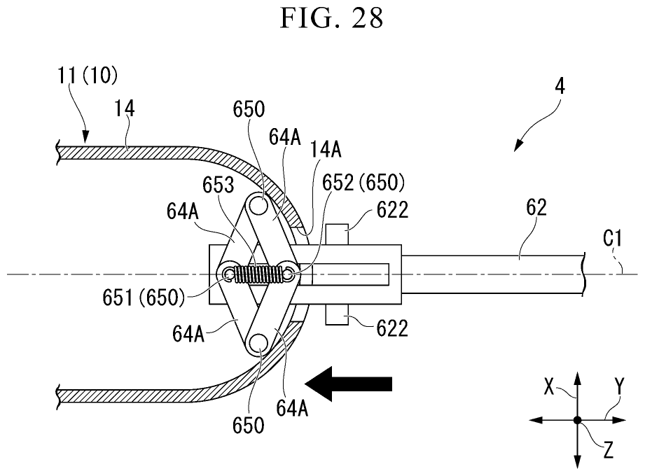

As a result, as shown in FIG. 28, when the link mechanism 64 climbs over the insertion port 14A and completely enters the arm member 11, the width of the link mechanism 64 in the radial direction is larger than the width of the insertion port 14A. Therefore, the link mechanism 64 is engaged with the insertion port 14A, and the movement of the link mechanism 64 toward the proximal end side is restricted by the insertion port 14A when no external force is applied.

By the above described operations, it is possible to make the arm member 11 and the operation wire 62 according to the present embodiment to engage with each other so as to attach the clip 10 to the applicator 30. Subsequently, the operator may treat (ligate) the target tissue using the medical device 4 similarly to the above-described embodiments.

According to the medical device 4 according to the present embodiment, during the procedures of treating the target tissue, the force by the operator to retract the slider toward the proximal end side and the biasing force by the elastic member 653 are applied in the same direction such that as shown in FIG. 28, even if the operator retracts the slider toward the proximal end side, it is impossible for the link mechanism 64 to be stretched in the longitudinal direction of the operation wire 62 and deformed. In other words, according to the medical device 4 according to the present embodiment it is possible to prevent the link mechanism 64 from being deformed and unintentionally slipping from the clip 10 during the procedures of treating the target tissue.

According to the medical device 4 according to the present embodiment, when the treatment on the target tissue is completed, the operator may retract the slider toward the proximal end side with a force larger than the maximum retraction force during the treatment procedures so as to make the insertion port 14A of the arm member 11 to be plastically deformed by the arm 64A of the link mechanism 64 and take out the link mechanism 64.

According to the medical device 4 according to the present embodiment, the elastic member 653 is extended to change the width of the operation wire 62 of the link mechanism 64 in the radial direction such that the link mechanism 64 passes through the insertion port 14A of the arm member 11, the operation wire 62 and the clip 10 are transitioned from the release configuration to the engagement configuration, and the clip 10 is engaged with the applicator 30.

According to the medical device 4 according to the present embodiment, it is possible to prevent the clip 10 from unintentionally slipping from the operation wire 62 during the procedures of treating the target tissue, similarly to the above embodiments.

### Fifth Embodiment

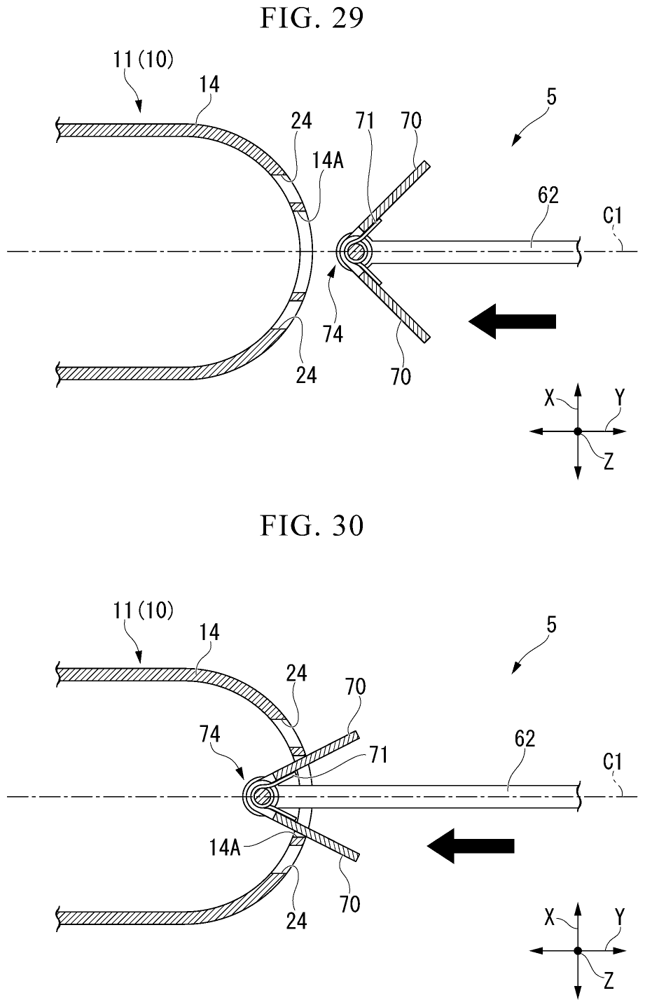

A medical device 5 according to a fifth embodiment of the present disclosure will be described below with reference to FIG. 29 to FIG. 33. Hereinafter, the same configurations as those of the medical device 1 according to the first embodiment described above will be designated to the same reference numerals, the description thereof will be omitted, and differences from the above embodiment will be mainly described.

FIG. 29 to FIG. 33 are views showing a structure in which the operation wire 62 and the clip 10 are engaged with each other and an operation of attaching the clip 10 to the applicator 30 in the medical device 5 according to the present embodiment.

As shown in FIG. 29, in the medical device 5 according to the present embodiment, a link mechanism 74 configured by a pair of legs 70 connected by an elastic member (spring hinge) 71 is provided at the distal end side of the operation wire 62. The elastic member 71 is configured to bias the pair of legs 70 in the direction in which the pair of legs 70 are separated from each other. In other words, in a natural state in which no external force is applied, the pair of legs 70 connected by the elastic member 71 are separated from each other with a certain distance (angle). The operation wire 62 is connected to the elastic member 71. Accordingly, the pair of legs 70 and the elastic member 71 may advance and retract together with the operation wire 62. According to the present embodiment, since the pair of legs 70 and the elastic member 71 have to be repeatedly used, it is preferable that the pair of legs 70 and the elastic member 71 be formed of a biocompatible metal material.

According to the present embodiment, the insertion port 14A formed in the intermediate portion 14 of the arm member 11 only have to be formed with a width to allow the pair of legs 70 provided at the distal end of the operation wire 62 to pass in a compressed state; however, the specific dimensions are not particularly limited. Two engagement holes 24 are formed in the inner circumferential surface of the intermediate portion 14 of the arm member 11 on both sides of the insertion port 14A. According to the present embodiment, the two engagement holes 24 are provided to engage the pair of legs 70.

### (Operation to Attach Clip to Applicator)

When the clip 10 according to the present embodiment is attached to the applicator 30, the operator pushes the slider of the operation unit on the proximal end side (not shown) toward the proximal end side to move the operation wire 62 and the link mechanism 74 toward the arm member 11 and toward the distal end side. According to the present embodiment, the distal ends of the pair of legs 70 are connected to the elastic member 71 respectively, and the distance between the pair of legs 70 sequentially increases toward the proximal end side. The width between the proximal ends of the pair of legs 70 is larger than the width of the insertion port 14A of the intermediate portion 14 of the arm member 11.

Since the link mechanism 74 according to the present embodiment has the above-described configuration, as shown in FIG. 30, after the elastic member 71 enters the insertion port 14A, the pair of legs 70 contact the edge of the insertion port 14A to be inserted into the insertion port 14A while the distance between the pair of legs 70 are decreased. During the process, the elastic member 71 is compressed.

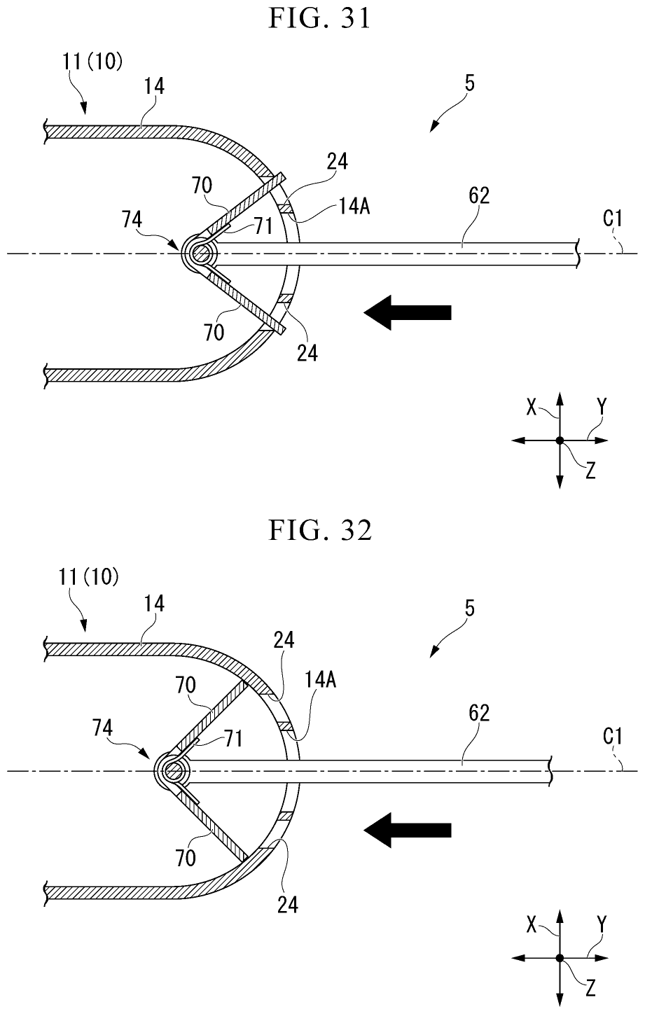

As shown in FIG. 31, when the pair of legs 70 completely pass through the insertion port 14A and enter the inside of the arm member 11, the elastic restoring force of the elastic member 71 restores the pair of legs 70 to be separated from each other. As a result, the pair of legs 70 slide into the engagement holes 24 formed in the vicinity of the insertion port 14A, so that the pair of legs 70 engage with the pair of engagement holes 24, respectively, as shown in FIG. 31. In this state, the pair of legs 70 and the arm member 11 are engaged with each other, and the operation wire 62 and the arm member 11 are connected. Since the pair of legs 70 are engaged with the pair of engagement holes 24 respectively, when the operator rotates the slider (not shown), the arm member 11 is rotated together with the pair of legs 70 connected to the distal end side of the operation wire 62. According to the present embodiment, both of the advancement/retraction and rotation of the operation wire 62 generated by the operator operating the slider that is not shown are transmitted to the arm member 11 via the link mechanism 74.

Accordingly, the operator may engage the clip 10 and the operation wire 62 with each other so as to attach the clip 10 to the applicator. Subsequently, the operator may treat (ligate) the target tissue using the medical device 4 similarly to the above-described embodiments.

According to the present embodiment, the elastic member 71 is configured to bias the pair of legs 70 in the direction in which the pair of legs 70 are separated from each other. Accordingly, as shown in FIG. 31, in the state in which the pair of legs 70 are engaged with the engagement holes 24 respectively, even the operator operates the slider (not shown) to retract the operation wire 62, it is impossible to unintentionally remove the pair of legs 70 from the engagement holes 24. In other words, in the process of treating the target tissue using the medical device 5 according to the present embodiment, even if the force for retracting the slider that is not shown to the proximal end side by the operator is applied to the operation wire 62 and the link mechanism 74, it is impossible to release the engagement between the link mechanism 74 and the arm member 11. Accordingly, according to the link mechanism 74 according to the present embodiment, it is possible to prevent the clip 10 from unintentionally slipping from the operation wire 62 in the process of treating the target tissue in the state in which the clip 10 is attached to the applicator 30.

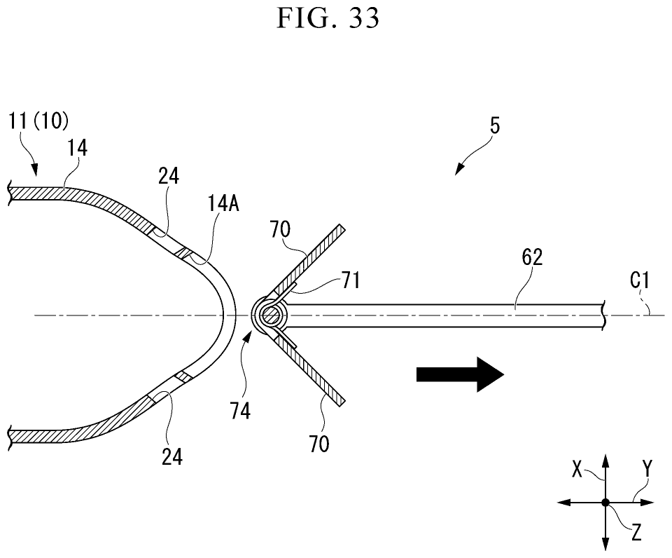

According to the medical device 5 according to the present embodiment, when the treatment on the target tissue is completed, the operator may push the slider (not shown) toward the distal end side, as shown in FIG. 32, to release the engagement between the pair of legs 70 of the link mechanism 74 and the pair of engagement holes 24. In this state, the pair of legs 70 of the link mechanism 74 are in contact with the arm member 11 at the intermediate portion 14 of the arm member 11 and the pair of legs 70 are caught thereby. When the operator retracts the slider (not shown) to the proximal end side, the force of this operation is applied to the operation wire 62 and the link mechanism 74, such that the pair of legs 70 of the link mechanism 74 press the intermediate portion 14 of the arm member 11. When the operator subsequently retracts the slider (not shown) to the proximal end side, the amount of force that the pair of legs 70 press the intermediate portion 14 increases. When the amount of the force that the pair of legs 70 press against the intermediate portion 14 exceeds the stress that the intermediate portion 14 of the arm member 11 can withstand, plastic deformation occurs in the intermediate portion 14 of the arm member 11 as shown in FIG. 33, and the link mechanism 74 may be removed from the insertion port 14A that is deformed and expanded.

According to the present embodiment, the strength of the elastic member 71 and the pair of legs 70 in the link mechanism 74 is set to be larger than the stress that the arm member 11 can withstand, such that the link mechanism 74 may be removed from the insertion port 14A of the arm member 11 without being deformed. According to the present embodiment, the stress that the intermediate portion 14 of the arm member 11 can withstand is set to be larger than the maximum force by the operator retracting the slider (not shown) in the treatment process such that it is possible to prevent unintentional deformation in the arm member 11.

According to the medical device 5 according to the present embodiment, the elastic member 71 is compressed and the opening width between the pair of legs 70 is decreased to allow the link mechanism 74 to pass through the insertion port 14A of the arm member 11 such that the operation wire 62 and the clip 10 may transition from the release configuration to the engagement configuration and the clip 10 may be engaged with the applicator 30.

According to the medical device 5 according to the present embodiment, similarly to the above-described embodiments, it is possible to prevent the clip 10 from unintentionally slipping from the operation wire 62 during the process of treating the target tissue.

Although the respective embodiments and modifications of the present disclosure have been described above, the technical scope of the present disclosure is not limited to the above-described embodiments, and configurations in the respective embodiments and modifications within the scope not departing from the spirit of the present disclosure. It is possible to change the combination of elements, make various changes to each configuration element, or delete each configuration element. For example, the configuration according to any one of above-described embodiments and modifications of the present disclosure may be appropriately combined with each modification of the operation portion. The present disclosure is not limited by the above description, but only by the appended claims.

For example, by appropriately combining the configuration of the applicator according to the first embodiment and the modification of the present disclosure and the connection structure for engaging the operation wire and the clip according to each embodiment of the present invention, it is possible to relegalize both goals of re-loading the treatment device and re-grasping the target tissue by the treatment device.

What is claimed is:

1. A medical device, comprising:
a clip that includes an arm member and a pressing tube configured to receive the arm member;
an operation wire configured to advance and retract to open and close the arm member;
a sheath configured to accommodate the operation wire; and
a link configured to connect the operation wire with the clip unit; wherein:
the link mechanism includes:
a convex portion configured to transition between a first configuration in which the convex portion has a first width and a second configuration in which the convex portion has a second width larger than the first width; and
an accommodation portion having an insertion port with an third width larger than the first width and smaller than the second width the convex portion is spaced apart from an edge of the insertion port to form a space between the convex portion and the edge of the insertion port when the convex portion is inserted into the insertion port, and
the link is configured to transition to the second configuration to cause the convex portion to engage with the accommodation portion when the convex portion passes through the insertion port in the first configuration.

2. The medical device according to claim 1, wherein the link is positioned inside the pressing tube or the sheath when the operation wire is advanced to a maximum amount.

3. The medical device according to claim 1, wherein the link mechanism contacts the sheath when the operation wire is advanced to a maximum amount.

4. The medical device according to claim 1, wherein:
the convex portion has a flange shape opening away from the longitudinal axis, and
the convex portion having the first width is configured to move such that the convex portion has the second width that is smaller than the first width; and
the insertion port has a thickness that is smaller than the first width of the convex portion and larger than the second width of the convex portion.

5. The medical device according to claim 1, wherein:
the link mechanism includes a block member with a width larger than the third width of the insertion port, and
the convex portion is configured to transition from the first configuration to the second configuration when the convex portion is inserted via the insertion port to engage with the block member.

6. An attachment method for a medical device, the medical device comprising:
a clip including an arm member and a pressing tube configured to receive the clip;
an operation wire configured to advance and retract to open and close the arm member;
an applicator having a sheath configured to accommodate an operation wire;
a convex portion provided at a distal end of the operation wire and configured to transition between a first configuration in which the convex portion has a first width in a predetermined direction around a longitudinal axis of the operation wire and a second configuration in which the convex portion has a second width in the predetermined direction, the second width being larger than the first width; and
an accommodation portion having an insertion port with an opening width larger than the first width and smaller than the second width in the predetermined direction around the longitudinal axis of the operation wire, the attachment method comprising:
approaching the sheath to the pressing tube;
moving the operation wire;
passing the convex portion through the insertion port of the accommodation portion when the convex portion is provided in the first configuration; and
transitioning the convex portion from the first configuration to the second configuration to engage with the accommodation portion.

7. The medical device according to claim 1, wherein when the convex portion is provided in the first configuration, the first width of the convex portion extends along a first direction perpendicular to the longitudinal axis, and the second width of the convex portion extends along a second direction perpendicular to the longitudinal direction and the first direction.

8. The medical device according to claim 7, wherein when the convex portion is provided in the second configuration, the first width of the convex portion extends along the second direction and the second width of the convex portion extends along the first direction.
